(19) United States
(12) Patent Application Publication
(10) Pub. No.: US 2025/0263715 A1
(43) Pub. Date: Aug. 21, 2025

(54) IMPROVED CHIMERIC AND ENGINEERED SCAFFOLDS AND CLUSTERS OF MULTIPLEXED INHIBITORY RNA

(71) Applicant: CELYAD Oncology S.A., Mont-Saint-Guibert (BE)

(72) Inventors: Eytan BREMAN, Mont-Saint-Guibert (BE); Mikhail STEKLOV, Mont-Saint-Guibert (BE); Matteo ROSSI, Mont-Saint-Guibert (BE)

(21) Appl. No.: 18/558,906

(22) PCT Filed: May 4, 2022
(86) PCT No.: PCT/EP2022/062064

§ 371 Date: Nov. 3, 2023

(30) Foreign Application Priority Data
May 4, 2021 (GB) 2106354.0

(51) Int. Cl. C12N 15/113; A61K 35/17; A61K 40/11; C12N 5/0783

(52) U.S. Cl. CPC C12N 15/1138; A61K 35/17; A61K 40/11; C12N 5/0636; C12N 2310/141; C12N 2310/3519; C12N 2310/531; C12N 2510/00

(57) ABSTRACT

The present application relates to the field of RNA interference, more particularly RNA interference as applied in immunotherapy, such as adoptive cell therapy (ACT). Here, chimeric clusters of multiple shRNA scaffolds, designed to downregulate multiple targets are proposed. Also proposed are polynucleotides, vectors comprising the shRNA and cells expressing such shRNAs, alone or in combination with a protein of interest such as a chimeric antigen receptor (CAR) or T cell receptor (TCR). These cells are particularly suitable for use in immunotherapy.

### FIELD OF THE INVENTION

The present application relates to the field of RNA interference, more particularly RNA interference as applied in immunotherapy, such as adoptive cell therapy (ACT). Here, chimeric clusters of multiple shRNA scaffolds, designed to downregulate multiple targets are proposed. Also proposed are polynucleotides, vectors comprising the shRNA and cells expressing such shRNAs, alone or in combination with a protein of interest such as a chimeric antigen receptor (CAR) or T cell receptor (TCR). These cells are particularly suitable for use in immunotherapy.

### BACKGROUND

Downregulating multiple targets simultaneously in hard to transduce cells in an efficient way is a known problem. Multiplex genome engineering methods often are cumbersome. When looking to solve the issues encountered with multiplexed genome engineering, systems could be considered that offer the possibility of a knockdown instead of a genetic knockout, which would lead to greater flexibility (e.g. temporal regulation would become possible). Ideally, these systems should also be less cumbersome (so that no separate proteins need to be engineered for each target, or so that downregulation can be achieved in a single transduction step), and should be sufficiently efficient and specific.

One solution that could be considered is RNA interference (RNAi). Several mechanisms of RNAi gene modulation exist in plants and animals. A first is through the expression of small non-coding RNAs, called microRNAs (“miRNAs”). miRNAs are able to target specific messenger RNAs (“mRNA”) for degradation, and thereby promote gene silencing.

Because of the importance of the microRNA pathway in the modulation of gene activity, researchers are currently exploring the extent to which small interfering RNAs (“siRNAs”), which are artificially designed molecules, can mediate RNAi. siRNAs can cause cleavage of a target molecule, such as mRNA, and similar to miRNAs, in order to recognize the target molecule, siRNAs rely on the complementarity of bases.

Within the class of molecules that are known as siRNAs are short hairpin RNAs (“shRNAs”). shRNAs are single stranded molecules that contain a sense region and an antisense region that is capable of hybridizing with the sense region. shRNAs are capable of forming a stem and loop structure in which the sense region and the antisense region form part or all of the stem. One advantage of using shRNAs is that they can be delivered or transcribed as a discreet single entity that can be incorporated either as a single unit or as a part of a multi-component system, none of which is reasonably possible when an siRNA has two separate strands. However, like other siRNAs, shRNAs still target mRNA based on the complementarity of bases.

Many conditions, diseases, and disorders are caused by the interaction between or among a plurality of proteins. Consequently, researchers are searching for effective ways to deliver multiple siRNAs to a cell or an organism at the same time.

One delivery option is the use of vector technologies to express shRNAs in the cells in which they will be processed through the endogenous miRNA pathway. The use of separate vectors for each shRNA can be cumbersome. Consequently, researchers have begun to explore the use of vectors that are capable of expressing a plurality of shRNAs. Unfortunately, the reported literature describes several challenges when expressing multiple shRNAs from a single vector. Among the issues that researchers have encountered are: (a) a risk of vector recombination and loss of shRNA expression; (b) reduced shRNA functionality by positional effects in a multiplex cassette (e.g. as a result of secondary structure); (c) the complexity of shRNA cloning; (d) RNAi processing saturation; (e) cytotoxicity; and (f) undesirable off-target effects.

Moreover, while siRNA has been shown to be effective for short-term gene inhibition in certain transformed mammalian cell lines, its use in primary cell cultures or for stable transcript knockdown proves more of a challenge. Knockdown efficacy is known to vary widely and ranges between <10% to >90% (e.g. Taxman et al., 2006), so further optimisation is necessary. As efficacy typically decreases when more than one inhibitor is expressed, this optimisation is even more important in such setting.

Therefore, there remains a need to develop efficient cassettes and vectors for delivery of multiplexed RNA interference molecules. While true for cellular applications in general, this is even less explored in the field of ACT, and there is a high need for efficient systems in these cells.

Thus, there is a need in the art to provide systems allowing cell therapy with multiplexed knockdown of targets that do not require multi-step production methods (and thus offer a comparative ease of manufacture and reduced costs), and offer flexibility (e.g. by making changes reversible, allowing attenuation of knockdown (e.g. to avoid toxicity), or swapping in one target for another).

### SUMMARY

Surprisingly, it is demonstrated herein that shRNA can not only successfully be multiplexed in cells, particularly in engineered immune cells, but multiple targets are also very efficiently downregulated, by making use of scaffolds, particularly multiplexed scaffolds, of the miR-17 miRNA family cluster (i.e. one of the miR-17˜92 paralogs), in particular scaffolds such as found in the miR-106a˜363 cluster, the miR-17˜92 cluster, the miR106b-25 cluster and combinations thereof, particularly chimeric combinations thereof. Chimeric combinations can be chimeric clusters (wherein scaffolds from these three different clusters are used to create a new cluster) or chimeric scaffolds (wherein at least the lower stem part of the scaffold is from a different miRNA than the upper stem and/or loop part), or a combination of both.

Accordingly, objects of the invention are provided in the following clauses:

1. A nucleic acid molecule comprising at least one RNA interference molecule with an engineered scaffold, wherein the engineered scaffold comprises a lower stem region and an upper stem/loop region, and wherein the lower stem region of the scaffold is that of a miR scaffold from the miR-17 family cluster, and wherein at least part of the upper stem/loop region of the scaffold has been engineered to differ from the wild-type/natural sequence.

2. The nucleic acid molecule according to clause 1, wherein the lower stem region of the engineered scaffold is selected from a miR-17 scaffold, a miR-18a scaffold, a miR-19a scaffold, a miR-20a scaffold, a miR-19b-1 scaffold, a miR-92-1 scaffold, a miR-106a scaffold, a miR-18b scaffold, a miR-20b scaffold, a miR-19b-2 scaffold, a miR-92-2 scaffold, a miR-363 scaffold, a miR-106b scaffold, a miR-25 scaffold, and a miR-93 scaffold.

3. The nucleic acid molecule according to clause 1 or 2, wherein the engineered scaffold is a chimeric scaffold and wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold.

4. A nucleic acid molecule according to any one of clause 1 to 3, wherein the at least one RNA interference molecule are at least two multiplexed RNA interference molecules.

5. A nucleic acid molecule comprising at least two RNA interference molecules with different scaffolds, wherein the at least two different scaffolds have a lower stem region of a miR scaffold from the miR-17 family cluster; and wherein
at least one RNA interference molecule has a chimeric scaffold wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold; and/or wherein
the scaffolds from the at least two RNA interference molecules are a miR-17 family scaffold from different miR-17 family clusters.

6. A vector suitable for expression in engineered immune cells comprising a nucleic acid molecule according to any one of clauses 1 to 5.

7. An engineered cell comprising:
a first exogenous nucleic acid molecule encoding a protein of interest, and
a second nucleic acid molecule comprising at least one RNA interference molecule with an engineered scaffold, wherein the lower stem region of the scaffold is that of a miR scaffold from the miR-17 family cluster, and wherein at least part of the upper stem/loop region of the scaffold has been engineered to differ from the wild-type/natural sequence.

8. An engineered cell comprising:
a first exogenous nucleic acid molecule encoding a protein of interest, and
a second nucleic acid molecule comprising at least two RNA interference molecules with different scaffolds, wherein the at least two different scaffolds have a lower stem region of a miR scaffold from the miR-17 family cluster; and wherein
   at least one RNA interference molecule has a chimeric scaffold wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold; and/or wherein
   the scaffolds from the at least two RNA interference molecules are a miR-17 family scaffold from different miR-17 family clusters.

9. The engineered cell of clause 7 or 8, which is an engineered immune cell.

10. The engineered immune cell of clause 9, wherein the immune cell is selected from a T cell, a NK cell, a NKT cell, a macrophage, a stem cell, a progenitor cell, and an iPSC cell.

11. The engineered cell of any one of clause 7 to 10, wherein the protein of interest is a receptor, particularly a chimeric antigen receptor or a TCR.

12. The engineered cell of any one of clauses 7 to 11, wherein the at least one RNA interference molecule is at least two multiplexed RNA interference molecules under control of one promoter.

13. The engineered cell of clause 12, wherein the at least two multiplexed RNA interference molecules are at least three multiplexed RNA interference molecules.

14. The nucleic acid molecule according to any one of clause 1 to 5, vector according to clause 6 or engineered cell of any one of clause 7 to 13, wherein the molecule targeted by the at least one RNA interference molecules is selected from: a MHC class I gene, a MHC class II gene, a MHC coreceptor gene (e.g. HLA-F, HLA-G), a TCR chain, NKBBiL, LTA, TNF, LTB, LST1, NCR3, AIF1, LY6, a heat shock protein (e.g. HSPA1L, HSPA1A, HSPA1B), complement cascade, regulatory receptors (e.g. NOTCH4), TAP, HLA-DM, HLA-DO, RING1, CD52, CD247, HCP5, B2M, MICA, MICB, ULBP1, ULBP2, ULBP3, ULBP4, ULBP5, ULBP6, 2B4, A2AR, BAX, BLIMP1, C160 (POLR3A), CBL-B, CCR6, CD7, CD27, CD28, CD38, CD95, CD96, CD123, CD272 (BTLA), CD276 (aka B7-H3), CIITA, CTLA4, DGK [DGKA, DGKB, DGKD, DGKE, DKGG, DGKH, DGKI, DGKK, DGKQ, DGKZ], DNMT3A, DR4, DR5, EGR2, FABP4, FABP5, FASN, GMCSF, HPK1, IL-10R [IL10RA, IL10RB], IL2, LAG3 (CD223), LFA1, NEAT 1, NFkB (including RELA, RELB, NFkB2, NFkB1, REL), NKG2A, NR4A (including NR4A1, NR4A2, NR4A3), PD1, PI3KCD, PPP2RD2, PRAS40, RAPTOR, SHIP1, SOAT1, SOCS1, T-BET, TCF7 (aka TCF-1), TET2, TGFBR1, TGFBR2, TGFBR3, TIGIT, TIM3 (aka HAVCR2 or CD366), TOX, TOX2, VISTA (aka VSIR or B7-H5), ZC3H12A (also known as regnase-1 or MCPIP) and ZFP36L2.

15. The nucleic acid molecule according to any one of clause 1 to 5, vector according to clause 6 or engineered cell of any one of clause 7 to 14 for use as a medicament.

16. The nucleic acid molecule according to any one of clause 1 to 5, vector according to clause 6 or engineered cell of any one of clause 7 to 14 for use in the treatment of cancer.

17. A method of treating cancer, comprising administering to a subject in need thereof a suitable dose of cells according to any one of clause 7 to 14, thereby improving at least one symptom.

18. The nucleic acid molecule according to any one of clause 1 to 5 or vector according to claim 6, wherein the lower stem region and/or up to 10 nucleotides adjacent to this region from 3′ and/or 5′ sides has been engineered to differ from the wild-type/natural sequence.

Accordingly, it is an object of the invention to provide vectors comprising nucleic acid sequences comprising at least one RNA interference molecule having a scaffold selected from one present in the miR-106a˜363 cluster, particularly with a scaffold selected from a miR-106a scaffold, a miR-18b scaffold, a miR-20b scaffold, a miR-19b-2 scaffold, a miR-92-2 scaffold, and a miR-363 scaffold. According to particular embodiments, the vectors are suitable for expression in eukaryotic cells, particularly in immune cells. The RNA interference molecules typically also contain a target sequence not present in the wild-type/natural scaffold sequence. Typically this is achieved by substituting the wild-type/naturally occurring target sequence in the microRNA scaffold (typically referred to as the mature sequence) with a target sequence of choice, e.g. a target sequence that matches a sequence of a mRNA encoding a target protein. Most particularly, the target sequence has a length of between 18-23 nucleic acids. The complement strand of the target sequence is typically referred to as the passenger sequence.

According to specific embodiments, at least one of the scaffolds of the one or more RNA interference molecules is a scaffold selected from a miR-106a scaffold, a miR-18b scaffold, and a miR-20b scaffold. In other words, according to these specific embodiments, vectors are provided comprising nucleic acid sequences encoding at least one RNA interference molecule with a scaffold selected from one present in the first three scaffolds of the miR-106a˜363 cluster, i.e. with a scaffold chosen from a miR-106a scaffold, a miR-18b scaffold, and a miR-20b scaffold. For instance, at least one RNA interference molecule can have a miR-106a scaffold, while other RNA interference molecules can have an independently selected scaffold, such as a scaffold independently selected from a miR-106a scaffold, a miR-18b scaffold, a miR-20b scaffold, a miR-19b-2 scaffold, a miR-92-2 scaffold, and a miR-363 scaffold.

According to particular embodiments, more than one RNA interference molecule will be present in the vector. According to these embodiments, the at least one RNA interference molecule then is at least two RNA interference molecules, particularly at least two multiplexed RNA interference molecules. Thus, according to these embodiments, vectors are provided comprising nucleic acid sequences encoding at least two RNA interference molecule having a scaffold selected from one present in the miR-106a˜363 cluster, miR-17˜92 cluster, and the miR106b-25 cluster. Particularly, at least one of the scaffolds will be chimeric (wherein the lower stem region of the scaffold is from a different miRNA scaffold than the upper stem and/or loop region of the scaffold), most particularly the upper stem and loop region of the scaffold is from a scaffold of the miR-17 family. According to alternative (but not exclusive) embodiments, the at least two RNA interference molecules have a scaffold selected from a miR-17 family scaffold from at least two different clusters selected from the miR-106a˜363 cluster, miR-17˜92 cluster, and the miR106b-25 cluster. In other words, the at least two RNA interference molecules have a scaffold selected from at least two of the following three groups: the miR-106a scaffold and miR-20b scaffold; the miR-17 scaffold and miR-20a scaffold; the miR-106b scaffold and miR-93 scaffold.

When at least two multiplexed RNA interference molecules are present, those two or more molecules can have identical or different scaffolds. However, it is particularly envisaged that no more than three of the scaffolds are identical, and even more particularly envisaged that no more than two identical scaffolds are used. This to avoid recombination between identical scaffold sequences (see Example 5). For this reason, it is particularly envisaged that chimeric clusters and/or clusters with chimeric scaffold sequences are used, as outlined above.

According to specific embodiments, the scaffolds present in the vector are exclusively selected from the fifteen scaffolds present in the miR-106a˜363 cluster, miR-17˜92 cluster, and the miR106b-25 cluster (i.e., selected from a miR-17 scaffold, a miR-18a scaffold, a miR-19a scaffold, a miR-20a scaffold, a miR-19b-1 scaffold, a miR-92-1 scaffold, a miR-106a scaffold, a miR-18b scaffold, a miR-20b scaffold, a miR-19b-2 scaffold, a miR-92-2 scaffold, a miR-363 scaffold, a miR-106b scaffold, a miR-25 scaffold, and a miR-93 scaffold). As outlined above, these can also be chimeric scaffolds, where the lower stem part is selected from one of these fifteen scaffolds, and the upper stem and loop part is selected from a miR-17 family scaffold (i.e., selected from a miR-17 scaffold, a miR-20a scaffold, a miR-20b scaffold, a miR-93 scaffold, a miR-106a scaffold, and a miR-106b scaffold). However, it is also envisaged that these are further combined with different scaffold sequences, particularly different unrelated sequences (to avoid recombination), such as the miR-196a2 sequence.

According to particular embodiments, a scaffold sequence may have been engineered to reduce the number of mismatches and/or bulges in the stem region. More particularly, if one of the scaffold sequences that is used is a miR-18b scaffold, the scaffold can have been engineered (and is modified compared to the wild-type/natural sequence) to reduce the number of mismatches and/or bulges in the stem region (see Example 3).

According to a further aspect, provided herein are engineered cells comprising a nucleic acid molecule comprising at least two RNA interference molecules with different scaffolds, wherein the at least two different scaffolds have a lower stem region of a miR scaffold from the miR-17 family cluster; and wherein at least one RNA interference molecule has a chimeric scaffold wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold; and/or wherein the scaffolds from the at least two RNA interference molecules are a miR-17 family scaffold from different miR-17 family clusters.

The RNA interference molecules typically also contain a target sequence not present in the wild-type/natural scaffold sequence. To this end, the mature sequence of the respective miRNA scaffold is substituted with a target sequence of choice. The target sequence typically has a length of between 18-23 nucleic acids. It is particularly envisaged that the target sequence is directed against a sequence occurring in the engineered cells, particularly a sequence of a target. I.e., the at least one RNA interference molecule has a sequence targeting (by means of base pair complementarity) a sequence in the engineered cell encoding a protein to be downregulated.

According to further embodiments, provided are engineered cells comprising:
A first exogenous nucleic acid molecule encoding a protein of interest
a second nucleic acid molecule comprising at least one RNA interference molecule with an engineered scaffold, wherein the lower stem region of the scaffold is that of a miR scaffold from the miR-17 family cluster, and wherein at least part of the upper stem/loop region of the scaffold has been engineered to differ from the wild type/natural sequence.

According to further embodiments, the engineered cells comprise:
A first exogenous nucleic acid molecule encoding a protein of interest;
A second nucleic acid molecule comprising at least two RNA interference molecules with different scaffolds, wherein the at least two different scaffolds have a lower stem region of a miR scaffold from the miR-17 family cluster; and wherein at least one RNA interference molecule has a chimeric scaffold wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold; and/or wherein the scaffolds from the at least two RNA interference molecules are a miR-17 family scaffold from different miR-17 family clusters.

It is to be understood that the first and second exogenous nucleic acid molecule can be provided as one vector. Alternatively, they can be provided as separate nucleic acid molecules.

According to particular embodiments, the at least one RNA interference molecule comprises a target sequence within the scaffold which is different from the wild-type/natural target sequence of the scaffold (i.e., different from the mature strand of the miRNA scaffold). The target sequence typically is between 18 and 23 nucleotides long. According to particular embodiments, the RNA interference molecule is directed against a target in the engineered cell through base pair complementarity of the target sequence.

When at least two multiplexed RNA interference molecules are present, those two or more molecules can have identical or different scaffolds. However, it is particularly envisaged that no more than three of the scaffolds are identical, and even more particularly envisaged that no more than two identical scaffolds are used. This to avoid recombination between identical scaffold sequences (see Example 5).

The engineered cells are particularly eukaryotic cells, more particularly engineered mammalian cells, more particularly engineered human cells. According to particular embodiments, the cells are engineered immune cells. Typical immune cells are selected from a T cell, a NK cell, a NKT cell, a macrophage, a stem cell, a progenitor cell, and an iPSC cell.

According to particular embodiments, the engineered cells further contain a nucleic acid encoding a protein of interest. Particularly, this protein of interest is a receptor, particularly a chimeric antigen receptor or a TCR. Chimeric antigen receptors or engineered TCRs can be directed against any target, typical examples include CD19, CD20, CD22, CD30, BCMA, B7H3, B7H6, NKG2D, HER2, HER3, GPC3, MUC1, MUC16, TAG72, but many more exist and are also suitable. According to particular embodiments, more than one protein of interest can be present. In such cases, the second (or further) protein can be a receptor, or can for instance be a cytokine, chemokine, hormone, antibody, histocompatibility antigen (e.g. HLA-E), a tag, or any other protein of therapeutic or diagnostic value, or allowing detection.

According to specific embodiments, the first and second nucleic acid molecule are present in one vector, such as a eukaryotic expression plasmid, a mini-circle DNA, or a viral vector (e.g. derived from a lentivirus, a retrovirus, an adenovirus, an adeno-associated virus, and a Sendai virus).

The at least two multiplexed RNA interference molecules can be at least three, at least four, at least five, at least six, at least seven, at least eight, at least nine, at least ten or even more molecules, depending on the number of target molecules to be downregulated and practical considerations in terms of co-expressing the multiplexed molecules. According to particular embodiments, at least three multiplexed RNA interference molecules are used. According to further particular embodiments, at least one of the at least three RNA interference molecules has a scaffold selected from a miR-106a scaffold and a miR-20b scaffold. According to alternative embodiments, at least one of the at least three RNA interference molecules has a scaffold selected from a miR-106a scaffold and a miR-18b scaffold.

According to particular embodiments, a scaffold sequence may have been engineered to reduce the number of mismatches and/or bulges in the stem region. More particularly, if one of the scaffold sequences that is used is a miR-18b scaffold, the scaffold can have been engineered (and is modified compared to the wild-type/natural sequence) to reduce the number of mismatches and/or bulges in the stem region (see Example 3).

A “multiplex” is a polynucleotide that encodes for a plurality of molecules of the same type, e.g., a plurality of siRNA or shRNA or miRNA. Within a multiplex, when molecules are of the same type (e.g., all shRNAs), they may be identical or comprise different sequences. Between molecules that are of the same type, there may be intervening sequences such as the linkers described herein. An example of a multiplex of the present invention is a polynucleotide that encodes for a plurality of tandem miRNA-based shRNAs. A multiplex may be single stranded, double stranded or have both regions that are single stranded and regions that are double stranded.

According to particular embodiments, the at least two multiplexed RNA interference molecules are under control of one promoter. Typically, this promoter is not a U6 promoter. This because this promoter is linked to toxicity, particularly at high levels of expression. For the same reason, one can consider to exclude H1 promoters (which are weaker promoters than U6) or even Pol III promoters in general (although they can be suitable in certain conditions). According to specific embodiments, the promoter is selected from a Pol II promoter, and a Pol III promoter. According to particular embodiments, the promoter is a wild-type/natural or synthetic Pol II promoter. According to particular embodiments, the promoter is a Pol II promoter selected from a cytomegalovirus (CMV) promoter, an elongation factor 1 alpha (EF1α) promoter (core or full length), a phosphoglycerate kinase (PGK) promoter, a composite beta-actin promoter with an upstream CMV IV enhancer (CAG promoter), a ubiquitin C (UbC) promoter, a spleen focus forming virus (SFFV) promoter, a Rous sarcoma virus (RSV) promoter, an interleukin-2 promoter, a murine stem cell virus (MSCV) long terminal repeat (LTR), a Gibbon ape leukemia virus (GALV) LTR, a simian virus 40 (SV40) promoter, and a tRNA promoter. These promoters are among the most commonly used polymerase II promoters to drive mRNA expression, generic house keeping gene promoters can be used as well.

According to particular embodiments, the at least two multiplexed RNA interference molecules can be shRNA molecules or miRNA molecules. Most particularly, they are miRNA molecules. A difference between shRNA molecules and miRNA molecules is that miRNA molecules are processed by Drosha, while conventional shRNA molecules are not (which has been associated with toxicity, Grimm et al., Nature 441:537-541 (2006)).

According to specific embodiments, the different miRNA molecules are under control of one promoter.

According to particular embodiments, at least two of the multiplexed RNA interference molecules are directed against the same target. Note that RNA interference molecules directed against the same target can still have a different scaffold sequence and/or a different target sequence. According to further specific embodiments, at least two of the multiplexed RNA interference molecules have identical scaffolds, but different target sequences. According to alternative specific embodiments, at least two of the multiplexed RNA interference molecules have different scaffolds but identical target sequences. According to specific embodiments, at least two of the multiplexed RNA interference molecules are identical.

According to alternative embodiments, all of the at least two multiplexed RNA interference molecules are different. According to further specific embodiments, all of the at least two multiplexed RNA interference molecules are directed against different targets. Note that RNA interference molecules directed against different targets can still have the same scaffold (but will have a different target sequence).

Any suitable molecule present in the engineered cell can be targeted by the instant RNA interference molecules. Typical examples of envisaged targets are: a MHC class I gene, a MHC class II gene, a MHC coreceptor gene (e.g. HLA-F, HLA-G), a TCR chain, a CD3 chain, NKBBIL, LTA, TNF, LTB, LST1, NCR3, AIF1, LY6, a heat shock protein (e.g. HSPA1L, HSPA1A, HSPA1B), complement cascade, regulatory receptors (e.g. NOTCH4), TAP, HLA-DM, HLA-DO, RING1, CD52, CD247, HCP5, B2M, MICA, MICB, ULBP1, ULBP2, ULBP3, ULBP4, ULBP5, ULBP6, 2B4, A2AR, BAX, BLIMP1, C160 (POLR3A), CBL-B, CCR6, CD7, CD27, CD28, CD38, CD95, CD96, CD123, CD272 (BTLA), CD276 (aka B7-H3), CIITA, CTLA4, DGK [DGKA, DGKB, DGKD, DGKE, DKGG, DGKH, DGKI, DGKK, DGKQ, DGKZ], DNMT3A, DR4, DR5, EGR2, FABP4, FABP5, FASN, GMCSF, HPK1, IL-10R [IL10RA, IL10RB], IL2, LAG3 (CD223), LFA1, NEAT 1, NFkB (including RELA, RELB, NFkB2, NFkB1, REL), NKG2A, NR4A (including NR4A1, NR4A2, NR4A3), PD1, PI3KCD, PPP2RD2, PRAS40, RAPTOR, SHIP1, SOAT1, SOCS1, T-BET, TCF7 (aka TCF-1), TET2, TGFBR1, TGFBR2, TGFBR3, TIGIT, TIM3 (aka HAVCR2 or CD366), TOX, TOX2, VISTA (aka VSIR or B7-H5), ZC3H12A (also known as regnase-1 or MCPIP) and ZFP36L2.

Particularly suitable constructs have been identified which are miRNA-based. Accordingly, provided are engineered cells comprising a polynucleotide comprising a microRNA-based shRNA encoding region, wherein said microRNA-based shRNA encoding region comprises sequences that encode:

One or more artificial miRNA-based shRNA nucleotide sequences, wherein each artificial miRNA-based shRNA nucleotide sequence comprises
a miRNA scaffold sequence,
an active or mature sequence, and
a passenger or star sequence, wherein within each artificial miRNA-based shRNA nucleotide sequence, the active sequence is at least 70% complementary to the passenger sequence.

According to particular embodiments, the active sequence is at least 80% complementary to the passenger sequence, and can be at least 90% complementary to the passenger sequence or more.

A particular advantage is that the instant miRNA-based shRNA nucleotide sequences can be multiplexed. Accordingly, provided are engineered cells comprising a polynucleotide comprising a multiplexed microRNA-based shRNA encoding region, wherein said multiplexed microRNA-based shRNA encoding region comprises sequences that encode:

Two or more artificial miRNA-based shRNA nucleotide sequences, wherein each artificial miRNA-based shRNA nucleotide sequence comprises
a miRNA scaffold sequence,
an active or mature sequence, and
a passenger or star sequence, wherein within each artificial miRNA-based shRNA nucleotide sequence, the active sequence is at least 70% complementary to the passenger sequence.

Both the active sequence and the passenger sequence of each of the artificial miRNA-based shRNA nucleotide sequences are typically between 18 and 40 nucleotides long, more particularly between 18 and 30 nucleotides, more particularly between 18 and 25 nucleotides, most particularly between 18 and 23 nucleotides long. The active sequence can also be 18 or 19 nucleotides long. Typically, the passenger sequence has the same length as the active sequence, although the possible presence of bulges means that they are not always identical in length.

Typically, these microRNA scaffold sequences are separated by linkers. According to particular embodiments, at least some of the 5′ and/or 3′ linker sequence is used with its respective scaffold.

Artificial sequences can e.g. be wild-type/naturally occurring scaffolds (e.g. a miR cluster or fragment thereof, such as the miR-106a˜363 cluster) wherein the endogenous miR sequences have been replaced by shRNA sequences engineered against a particular target, can be repeats of a single miR scaffold (such as e.g. the miR-20b scaffold) wherein the endogenous miR sequences have been replaced by shRNA sequences engineered against a particular target, can be chimeric sequences combining elements (such as lower stem, upper stem and loop regions) from two different miRNA scaffolds, artificial miR-like sequences, or a combination thereof.

This engineered cell typically further comprises a nucleic acid molecule encoding a protein of interest, such as a chimeric antigen receptor or a TCR, and can be an engineered immune cell, as described above.

The expression of the at least one RNA interference molecule or co-expression of the multiplexed RNA interference molecules results in the suppression of at least one gene, but typically a plurality of genes, within the engineered cells. This can contribute to greater therapeutic efficacy.

The engineered cells described herein are also provided for use as a medicament. According to specific embodiments, the engineered cells are provided for use in the treatment of cancer.

This is equivalent as saying that methods of treating cancer are provided, comprising administering to a subject in need thereof a suitable dose of engineered cells as described herein, thereby improving at least one symptom.

The engineered cells may be autologous immune cells (cells obtained from the patient) or allogeneic immune cells (cells obtained from another subject).

### BRIEF DESCRIPTION OF THE FIGURES

FIG. 1: Schematic representation of clustered scaffolds, with indication of regions such as target sequence, upper stem, lower stem and scaffold.

FIG. 2: Shows the design of CAR expression vector (e.g. CD19, BCMA, B7H3, B7H6, NKG2D, HER2, HER3, GPC3) without (top) or with (below) an integrated miRNA scaffold, allowing for the co-expression of a CAR and multiple shRNAs (e.g. 2, 4, 6, 8, . . . ) from the same vector. LTR: Long terminal repeat; promoter (e.g. EF1α, PGK, SFFV, CAG, . . . ); a marker protein (e.g. truncated CD34, CD19); multiplexed shRNAs.

FIG. 3: Use of wild-type/natural mRNA Clusters increases the transduction efficiency as compared to repeated engineered single scaffolds. T cells were transduced with different vectors encoding a CD19 CAR and 3 to 6 multiplexed scaffolds according to the design shown in FIG. 2. CD34 was used as the reporter gene, and the % of CD34+ T cells at day 4 after transduction, as measured by FACS, is shown in the bottom panel. The top panel shows the same, but after purification (amount of cells eluted from the purification column divided on the amount of cells loaded on the purification column). 1-2: scaffolds from the miR-17-92 cluster, respectively 4 (miR-19a, miR-20a, miR-19b1, miR-92a1) and 3 scaffolds (miR-19a, miR-20a, miR-19b1); 3-5: scaffolds from the miR-106a-363 cluster, respectively 6 (all), 3 (the last 3) and 4 (the last 4); 6: all 3 scaffolds from the 106b-25 cluster; 7: all 3 scaffolds from the miR-23a˜27a˜24-2 cluster; 8-9: respectively 4 and 3 repeats of the miR-196a2 scaffold sequence; 10: mock vector with only the CD34 tag. Target genes included in the constructs were B2M, CD52 and CD247 for the triplex scaffolds, TRAC as additional gene in the tetraplex scaffolds. The hexaplex scaffold targeted each target gene twice, using two different target sequences for each target.

FIG. 4: Comparison of knockdown of CD247 (CD3zeta) between the 23a˜27a˜24-2 cluster and the miR-106a-363 cluster, as evaluated by TCR expression by FACS. 1: mock vector with only the CD34 tag; 2: all 3 scaffolds from the miR-23a˜27a˜24-2 cluster (CD247 target sequence in the miR-24-2 scaffold); 3-5: scaffolds from the miR-106a-363 cluster, respectively 6 (all), 3 (the last 3) and 4 (the last 4). CD247 target sequence is in the miR-363 scaffold; in 3, an additional different sequence is included in the miR-20b scaffold.

FIG. 5: Shows the miRNA 106a-363 cluster and design of constructs used for FIG. 6.

FIG. 6: Shown is RNA expression in primary T cells from a healthy donor transduced with retroviral vector encoding a second generation CD19-directed CAR, a truncated CD34 selection marker along with 3×shRNAs or 6×shRNAs targeting CD247, B2M or CD52, introduced in the 106a-363miRNA cluster. No shRNA (tCD34) was used as control. Two days after transduction, cells were enriched using CD34-specific magnetic beads, and further amplified in IL-2 (100 IU/mL) for 6 days. mRNA expression of CD247, B2M and CD52 was assessed by qRT-PCR using cyclophilin as house-keeping gene.

FIG. 7: comparison of different shRNA target sequences to allow finetuning of knockdown levels. Twelve different target sequences, all directed against CD247, were evaluated in the miR-20b scaffold. T cells were harvested at day 12 after activation (day 10 after transduction). TCRab levels were measured by FACS: MFI is presented as bar graphs. All shRNAs achieved at least 50% knockdown, several were much more efficient.

FIG. 8: Knockdown of CD95 in the miR-18b scaffold. Shown is a selected sequence out of 31 different target sequences, all directed against CD95, that were evaluated in the miR-18b scaffold. T cells were harvested at day 16 after activation (day 14 after transduction). CD95 levels were measured by FACS: MFI is presented as bar graphs. The most efficient shRNA achieved about 30% knockdown.

FIG. 9: Comparison of miR-106a, miR-18b and miR-20b scaffold structure. Target sequence (here a length of 20 bp) and a passenger strand are indicated as a rectangle. Whereas miR-106a and miR-20b have a mismatch at position 18 of the scaffold (position 14 of the target sequence), the scaffold of miR-18b is larger, and there are mismatches at positions 6, 11 and 15 of the target sequence (indicated with arrows 2, 3 and 4 respectively), as well as a bulge of 2 nucleic acids in the passenger strand between position 1 and 2 of the target sequence (indicated with arrow 1).

FIG. 10: Modifications of the miR-18b scaffold improve knockdown efficiency. FIG. 10A shows the modifications made to the miR-18b scaffold: removal of the bulge, removal of the individual mismatches, and removal of the bulge and the first two mismatches. FIG. 10B shows the effect of knockdown of CD95 in these miR-18b scaffolds: any construct that has a mismatch or bulge less compared to the wild-type/natural sequence achieves higher knockdown efficiency. Knockdown is measured in same way as in FIG. 8.

FIG. 11: Evaluation of target sequence length. Both for target sequences against B2M (left panel) and CD247 (right panel), the effect of target sequence length was evaluated on knockdown efficiency. Constructs are sometimes labelled with two lengths (19-20, 21-22 or 22-23) because the wild-type/natural scaffold sequence is identical to the target sequence at that position. Results shown are for the miR-106a scaffold, similar results were obtained for the miR-20b scaffold (not shown). Cluster: control with irrelevant sequence; as additional control the target sequence against respectively CD247 and B2M was used.

FIG. 12A-C: evaluation of simultaneous knockdown of different genes using different permutations of scaffolds. A: FACS data showing expression of B2M/HLA (left panel) and CD247/CD3zeta (right panel) for the duplex and triplex scaffolds indicated. B: MFI of FACS data of panel A, here including expression of CD95 for the triplex scaffolds. C: MFI of FACS data showing expression of B2M, CD247 and CD95 for the indicated constructs.

FIG. 13: Evaluation of changes in scaffolds and their effects on KD efficacy. A) Flow cytometry data of four different CAR T cells containing no shRNA (top), duplex shRNA against B2M (in miR-106a scaffold) and CD3 zeta (in miR-20b scaffold) (second from the top), triplex of miR-106a, miR-18b and miR-20b scaffolds containing a loop variant in scaffold miR-18b and a similar triplex wherein the upper stem/loop region of scaffold miR-18b was replaced with the upper stem/loop region of miR-17 (bottom). Expression of HLA-I (left panel), CD95 (middle) or TCR expression (right panel) are shown.

FIG. 14: Evaluation of target sequence changes when the multiplex shows stable microprocessing. A) Flow cytometric expression of shRNA targets in BCMA CAR T cells transduced without an shRNA (top), duplex against CD3zeta and B2M or triplex (B2M, CD3zeta, CD95) containing different target sequences of B2M. Left panel shows expression of HLA class I, middle panel of CD95 and right panel expression of TCR. B) Normalized values against the no shRNA arm of the mean fluorescence intensity of each target protein.

FIG. 15: Functional assessment of the different shRNA targets when knocked down by a chimeric triplex scaffold. A) shRNA against B2M protects BCMA CAR T cells against NK mediated killing in comparison to B2M knockout with Crispr cas9. B) shRNA against B2M inhibits T cell allorecognition similar to B2M knockout. C) shRNA against CD95 protects against FasL mediated apoptosis.

FIG. 16: Knockdown of target genes using a chimeric fourplex shRNA cluster containing 4 shRNA scaffolds derived from the three different miR17-92 paralogue clusters. A) diagram of the fourplex shRNA construct. B) Flow cytometric expression profile of a BCMA CAR T cell containing no shRNA (top histograms), a BCMA CAR T containing duplex shRNA (target antigens: B2M, CD3zeta) and the Fourplex (containing targets: B2M, CD28, MICA, CD3zeta). C) Relative expression of MICA as measured by qPCR.

FIG. 17: Knockdown of target genes using a chimeric fiveplex shRNA clusters containing 5 shRNA scaffolds derived from the three different miR17-92 paralogue clusters. A) Flow cytometric expression profile of a BCMA CAR T cells containing no shRNA (top histograms), a BCMA CAR T containing a fiveplex shRNA with unoptimized target sequences duplex (Fiveplex 1) and two Fiveplex clusters with the same optimized target sequences but in different orders (shRNA target antigens: B2M, CD3zeta, CD28, MICA, CD95). B) Relative expression of MICA as measured by qPCR. Fp1,2,3: Fiveplex 1,2,3. No sh: construct with CAR T, but no shRNA present.

FIG. 18: Knockdown of target genes using a fiveplex chimeric construct. A) diagram of the shRNA Fiveplex. B) Flow cytometric expression comparing a anti-BCMA CAR T cell without any shRNA (lower histograms), with triplex containing shRNA (targets: B2M, CD95, CD3zeta) and fiveplex containing shRNA (targets: B2M, CD95, CD3zeta, MICA, CD28; middle and top histograms respectively). C) Relative percentage of inhibition, normalized to the no shRNA arm using the mean fluorescence intensity.

FIG. 19: Knockdown of target genes using a sixplex chimeric construct. A) Flow cytometric expression comparing a anti-BCMA CAR T cell without any shRNA (lower histograms) and sixplex containing shRNA (upper histograms) (targets from left to right: CD38, B2M, CD95, CD3zeta, CD28, CD27). B) Relative percentage of inhibition, normalized to the no shRNA arm using the mean fluorescence intensity.

### DETAILED DESCRIPTION

### Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention.

Unless specifically defined herein, all terms used herein have the same meaning as they would to one skilled in the art of the present invention. Practitioners are particularly directed to Green and Sambrook, Molecular Cloning: A Laboratory Manual, 4th ed., Cold Spring Harbor Laboratory Press, New York (2012); and Ausubel et al., Current Protocols in Molecular Biology (up to Supplement 114), John Wiley & Sons, New York (2016), for definitions and terms of the art. The definitions provided herein should not be construed to have a scope less than understood by a person of ordinary skill in the art.

An “engineered cell” as used herein is a cell that has been modified through human intervention (as opposed to naturally occurring mutations).

The term “nucleic acid molecule” synonymously referred to as “nucleotides” or “nucleic acids” or “polynucleotide” as used herein refers to any polyribonucleotide or polydeoxyribonucleotide, which may be unmodified RNA or DNA or modified RNA or DNA. Nucleic acid molecules include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A “vector” is a replicon, such as plasmid, phage, cosmid, or virus in which another nucleic acid segment may be operably inserted so as to bring about the replication or expression of the segment. A “clone” is a population of cells derived from a single cell or common ancestor by mitosis. A “cell line” is a clone of a primary cell that is capable of stable growth in vitro for many generations. In some examples provided herein, cells are transformed by transfecting the cells with DNA.

As used herein, “to differ” or “differs” in terms of sequence, and particularly “to differ from a wild-type sequence” means that a sequence is altered compared to the wild-type/natural sequence, either by substitution, deletion and/or insertion of nucleic acids. Particularly in the context of differences in an upper stem/loop region, “to differ” of “differs” can mean that at least one mismatch or bulge has been removed or introduced compared to the wildtype sequence. If no mismatch or bulge has been removed or introduced, a sequence is considered to differ from the wild-type sequence if it has less than 98% sequence identity, less than 95% sequence identity, particularly less than 90% sequence identity, over its relevant length. In the context of upper stem/loop sequences, its relevant length is the length of the upper stem/loop region. Note that in the context of chimeric sequences, where the upper stem/loop sequence has been substituted, the appropriate wild-type sequence to compare to is that of the original scaffold (i.e., the scaffold corresponding to the lower stem region). A sequence that has been “engineered to differ” means that the change has been introduced purposefully, typically to achieve more desirable results (such as improved downregulation of a target sequence or improved microprocessing of a scaffold).

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications.

The term “exogenous” as used herein, particularly in the context of cells or immune cells, refers to any material that is present and active in an individual living cell but that originated outside that cell (as opposed to an endogenous factor). The phrase “exogenous nucleic acid molecule” thus refers to a nucleic acid molecule that has been introduced in the (immune) cell, typically through transduction or transfection. The term “endogenous” as used herein refers to any factor or material that is present and active in an individual living cell and that originated from inside that cell (and that are thus typically also manufactured in a non-transduced or non-transfected cell).

“Isolated” as used herein means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids.

“Multiplexed” as used herein in the context of molecular biology refers to the simultaneous targeting of two or more (i.e. multiple) related or unrelated targets. The term “RNA interference molecule” as used herein refers to an RNA (or RNA-like) molecule that inhibits gene expression or translation, by neutralizing targeted mRNA molecules. A RNA interference molecule neutralizes targeted mRNA molecules by base pair complementarity: within the RNA interference molecule is a target sequence (typically of 18-23 nucleic acids) that can hybridize to a targeted nucleic acid molecule. Examples include siRNA (including shRNA) or miRNA molecules. “Multiplexed RNA interference molecules” as used herein thus are two or more molecules that are simultaneously present for the concomitant downregulation of one or more targets. Typically, each of the multiplexed molecules will be directed against a specific target, but two molecules can be directed against the same target (and can even be identical).

A “promoter” as used herein is a regulatory region of nucleic acid usually located adjacent to a gene region, providing a control point for regulated gene transcription.

A “multiplex” is a polynucleotide that encodes for a plurality of molecules of the same type, e.g., a plurality of siRNA or shRNA or miRNA. Within a multiplex, when molecules are of the same type (e.g., all shRNAs), they may be identical or comprise different sequences. Between molecules that are of the same type, there may be intervening sequences such as the linkers described herein. An example of a multiplex of the present invention is a polynucleotide that encodes for a plurality of miRNA-based shRNAs. A multiplex may be single stranded, double stranded or have both regions that are single stranded and regions that are double stranded.

A “chimeric antigen receptor” or “CAR” as used herein refers to a chimeric receptor (i.e. composed of parts from different sources) that has at least a binding moiety with a specificity for an antigen (which can e.g. be derived from an antibody, a receptor or its cognate ligand) and a signaling moiety that can transmit a signal in an immune cell (e.g. a CD3 zeta chain. Other signaling or cosignaling moieties can also be used, such as e.g. a Fc epsilon RI gamma domain, a CD3 epsilon domain, the recently described DAP10/DAP12 signaling domain, or domains from CD28, 4-1BB, OX40, ICOS, DAP10, DAP12, CD27, and CD2 as costimulatory domain). A “chimeric NK receptor” is a CAR wherein the binding moiety is derived or isolated from a NK receptor.

A “TCR” as used herein refers to a T cell receptor. In the context of adoptive cell transfer, this typically refers to an engineered TCR, i.e. a TCR that has been engineered to recognize a specific antigen, most typically a tumor antigen. An “endogenous TCR” as used herein refers to a TCR that is present endogenously, on non-modified cells (typically T cells). The TCR is a disulfide-linked membrane-anchored heterodimeric protein normally consisting of the highly variable alpha (α) and beta (β) chains expressed as part of a complex with the invariant CD3 chain molecules. The TCR receptor complex is an octomeric complex of variable TCR receptor α and β chains with the CD3 co-receptor (containing a CD3γ chain, a CD3δ chain, and two CD3ε chains) and two CD3 ζ chains (aka CD247 molecules). The term “functional TCR” as used herein means a TCR capable of transducing a signal upon binding of its cognate ligand. Typically, for allogeneic therapies, engineering will take place to reduce or impair the TCR function, e.g. by knocking out or knocking down at least one of the TCR chains. An endogenous TCR in an engineered cell is considered functional when it retains at least 50%, at least 60%, at least 70%, at least 75%, at least 80%, or even at least 90% of signalling capacity (or T cell activation) compared to a cell with endogenous TCR without any engineering. Assays for assessing signalling capacity or T cell activation are known to the person skilled in the art, and include amongst others an ELISA measuring interferon gamma. According to alternative embodiments, an endogenous TCR is considered functional if no engineering has taken place to interfere with TCR function.

The term “immune cells” as used herein refers to cells that are part of the immune system (which can be either the adaptive or the innate immune system). Immune cells as used herein are typically immune cells that are manufactured for adoptive cell transfer (either autologous transfer or allogeneic transfer). Many different types of immune cells are used for adoptive therapy and thus are envisaged for use in the methods described herein. Examples of immune cells include, but are not limited to, T cells, NK cells, NKT cells, lymphocytes, dendritic cells, myeloid cells, macrophages, stem cells, progenitor cells or iPSCs. The latter three are not immune cells as such, but can be used in adoptive cell transfer for immunotherapy (see e.g. Jiang et al., Cell Mol Immunol 2014; Themeli et al., Cell Stem Cell 2015). Typically, while the manufacturing starts with stem cells or iPSCs (or may even start with a dedifferentiation step from immune cells towards iPSCs), manufacturing will entail a step of differentiation to immune cells prior to administration. Stem cells, progenitor cells and iPSCs used in manufacturing of immune cells for adoptive transfer (i.e., stem cells, progenitor cells and iPSCs or their differentiated progeny that are transduced with a CAR as described herein) are considered as immune cells herein. According to particular embodiments, the stem cells envisaged in the methods do not involve a step of destruction of a human embryo.

Particularly envisaged immune cells include white blood cells (leukocytes), including lymphocytes, monocytes, macrophages and dendritic cells. Particularly envisaged lymphocytes include T cells, NK cells and B cells, most particularly envisaged are T cells. In the context of adoptive transfer, note that immune cells will typically be primary cells (i.e. cells isolated directly from human or animal tissue, and not or only briefly cultured), and not cell lines (i.e. cells that have been continually passaged over a long period of time and have acquired homogenous genotypic and phenotypic characteristics). According to specific embodiments, immune cells will be primary cells (i.e. cells isolated directly from human or animal tissue, and not or only briefly cultured) and not cell lines (i.e. cells that have been continually passaged over a long period of time and have acquired homogenous genotypic and phenotypic characteristics). According to alternative specific embodiments, the immune cell is not a cell from a cell line.

A “microRNA scaffold”, “miRNA scaffold” or even “scaffold” as used herein refers to a well-characterized primary microRNA sequence containing specific microRNA processing requirements, wherein a RNA sequence can be inserted (typically to replace existing miRNA sequence with a siRNA directed against a specific target). A microRNA scaffold minimally consists of a double stranded upper stem region (typically of 18-23 nucleotides), with both sides of the stem region connected by a flexible loop sequence, and the upper stem region typically being processed by Dicer. Typically, the microRNA scaffold further comprises a lower stem region, and optionally it further comprises 5′ and 3′ flanking sequences or basal segments. The guide sequence or target sequence is inserted in the upper stem region and is a single strand sequence of 18-23 nucleotides. The target sequence recognizes its target through complimentary base pairing, so this sequence is typically identical to a sequence present in a target or its regulatory regions. A “target” or “target protein” as used herein refers to a molecule (typically a protein, but it can be a nucleic acid molecule) to be downregulated (i.e., of which the expression should be reduced in a cell). Note that miRNA works at the nucleic acid level, so even if it is directed against a protein, the miRNA target sequence will be identical to a sequence encoding the protein (e.g. a mRNA sequence) or to a sequence regulating expression of the protein (such as e.g. a 3′ UTR region).

Examples of a miRNA scaffold include e.g. scaffolds present in wild-type/naturally occurring miRNA clusters such as miR-106a, miR-18b, miR-20b, miR-19b-2, miR-92-2 or miR-363, or engineered scaffolds such as the SMARTvector™ micro-RNA adapted scaffold (Horizon Discovery, Lafayette, CO, USA). “miR-106a” as used herein corresponds to Gene ID 406899 in humans, “miR-18b” corresponds to Gene ID 574033 in humans, “miR-20b” corresponds to Gene ID 574032 in humans, “miR-19b-2” corresponds to Gene ID 406981 in humans, “miR-92-2” also known as “miR-92a-2” corresponds to Gene ID 407049 in humans, “miR-363” corresponds to Gene ID 574031 in humans.

A “microRNA cluster” or “miRNA cluster” as used herein refers to a collection of microRNA scaffolds that function together. These can be wild-type/naturally occurring clusters, or can be a combination of miRNA scaffolds that are not naturally found together. Wild-type/naturally occurring microRNA clusters are well described and include e.g. the miR-106a˜363 cluster, the miR-17˜92, miR-106b˜25, and miR-23a˜27a˜24-2 cluster. A miRNA cluster can be regarded as a combined scaffold. Thus, a cluster or “combined miRNA scaffold” as used herein refers to the combination of more than one miRNA scaffold to function under control of one promoter. The more than one miRNA scaffold can be identical or different, with target sequences directed against identical or different target proteins, and, if identical targets, with identical or different target sequences against that target. Such combined scaffold, when under control of one promoter, is also referred to as a “multiplex scaffold”, “multiplexed scaffold” or “multiplex miRNA scaffold”. Sometimes, when the number of scaffolds is determined, this can be used instead of the ‘multi-’prefix. E.g. a “duplex scaffold” means that two scaffolds are present, a “triplex scaffold” has three scaffolds, a “tetraplex” or “quadruplex” four, a “pentaplex” five, a “hexaplex” six, and so forth. In this way, a miRNA cluster with six different miRNA scaffolds (such as the wild-type/naturally occurring miR-106a-363 cluster) can be considered to be a hexaplex miRNA scaffold or a cluster of 6 miRNAs.

As used herein, a “miR-17 family cluster” is one of three paralogue miRNA clusters that contain (amongst others) scaffolds from the miR-17 family: the miR-106a˜363 cluster (consisting of (in order) the miR-106a scaffold, the miR-18b scaffold, the miR-20b scaffold, the miR-19b-2 scaffold, the miR-92a2 scaffold, and the miR-363 scaffold), the miR-17˜92 cluster (consisting of (in order) the miR-17 scaffold, the miR-18a scaffold, the miR-19a scaffold, the miR-20a scaffold, the miR-19b-1 scaffold and the miR-92-1 (also miR-92a1) scaffold), and the miR-106b˜25 cluster (consisting of (in order) the miR-106b scaffold, the miR-93 scaffold and the miR-25 scaffold). The “miRNA-17 family” or “miR-17 family” is grouped according to seed sequence and contains miR-17, miR-20a, miR-106a, mir-20b, miR-106b and miR-93. Likewise, other families grouped according to seed sequence are the “miR-18 family” (miR-18a, miR-18b), the “miR-19 family” (miR-19a, miR-19b-1 and miR-19b-2), and the “miR-92 family” (miR-92a1, miR-92a2, miR-363 and miR-25).

Thus, a “scaffold from a miR-17 family cluster” is any scaffold selected from the miR-17 scaffold, miR-18a scaffold, miR-18b scaffold, miR-19a scaffold, miR-19b-1 scaffold, miR-19b-2 scaffold, miR-20a scaffold, miR-20b scaffold, miR-25 scaffold, miR-92-1 scaffold, miR-92a2 scaffold, miR-93 scaffold, miR-106a scaffold, miR-106b scaffold, and miR-363 scaffold.

Whereas a “miR-17 family scaffold” as used herein is selected from the more limited group of six scaffolds from the miR-17 family: miR-17 scaffold, miR-20a scaffold, mir-20b scaffold, miR-93 scaffold, miR-106a scaffold, and miR-106b scaffold.

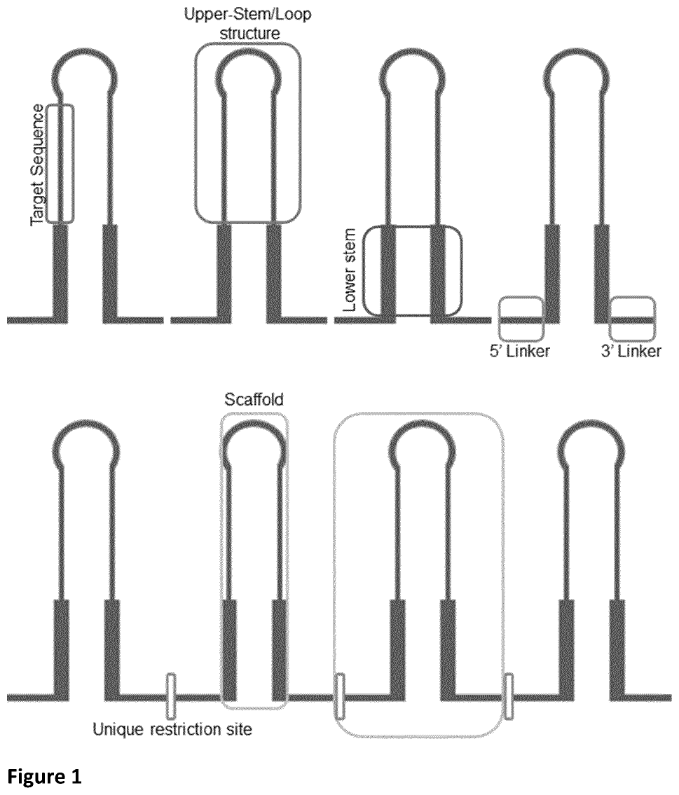

FIG. 1 shows schematic examples of multiplexed scaffold sequences, with indications of upper and lower stem regions, target sequences, individual scaffold, as used herein.

The term “subject” refers to human and non-human animals, including all vertebrates, e.g., mammals and non-mammals, such as non-human primates, mice, rabbits, sheep, dogs, cats, horses, cows, chickens, amphibians, and reptiles. In most particular embodiments of the described methods, the subject is a human.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival.

The treatment may be assessed by objective or subjective parameters; including the results of a physical examination, neurological examination, or psychiatric evaluations.

The phrase “adoptive cellular therapy”, “adoptive cell transfer”, or “ACT” as used herein refers to the transfer of cells, most typically immune cells, into a subject (e.g. a patient). These cells may have originated from the subject (in case of autologous therapy) or from another individual (in case of allogeneic therapy). The goal of the therapy is to improve immune functionality and characteristics, and in cancer immunotherapy, to raise an immune response against the cancer. Although T cells are most often used for ACT, it is also applied using other immune cell types such as NK cells, lymphocytes (e.g. tumor-infiltrating lymphocytes (TILs)), dendritic cells and myeloid cells.

An “effective amount” or “therapeutically effective amount” refers to an amount effective, at dosages and for periods of time necessary, to achieve a desired therapeutic result. A therapeutically effective amount of a therapeutic, such as the transformed immune cells described herein, may vary according to factors such as the disease state, age, sex, and weight of the individual, and the ability of the therapeutic (such as the cells) to elicit a desired response in the individual. A therapeutically effective amount is also one in which any toxic or detrimental effects of the therapeutic are outweighed by the therapeutically beneficial effects.

The phrase “graft versus host disease” or “GvHD” refers to a condition that might occur after an allogeneic transplant. In GvHD, the donated bone marrow, peripheral blood (stem) cells or other immune cells view the recipient's body as foreign, and the donated cells attack the body. As donor immunocompetent immune cells, such as T cells, are the main driver for GvHD, one strategy to prevent GvHD is by reducing (TCR-based) signaling in these immunocompetent cells, e.g. by directly or indirectly inhibiting the function of the TCR complex.

To assess whether the targeting of multiple genes in the context of adoptive cell transfer (ACT) is feasible without the need for genome editing (and its associated cost and complex manufacturing process), it was decided to test multiplexed RNA interference molecules.

The underlying approach is based upon the transcription of RNA from a specific vector that is processed by endogenous RNA processing machinery to generate an active shRNA which is able to target a mRNA of choice through base recognition and resultant destruction of that specific mRNA by the RISC complex. The specific destruction of the targeted mRNA results in the consequential reduction in expression of the relevant protein. Whilst RNA oligonucleotides can be transfected into target cells of choice to achieve a transient knockdown of gene expression, the expression of the desired shRNA from an integrated vector enables the stable knockdown of gene expression.

The successful expression of shRNA has largely been dependent upon coupling with a polymerase III (Pol III) promoter (e.g. H1, U6) that generate RNA species lacking a 5′ cap and 3′ polyadenylation, enabling processing of the shRNA duplex. Once transcribed, the shRNA undergoes processing, export from the nucleus, further processing and loading into the RNA-induced silencing complex (RISC) complex leading to the targeting degradation of mRNA of choice (Moore et al., 2010). Whilst effective, the efficiency of transcription driven by PolIII promoters can lead to cellular toxicity through the saturation of the endogenous microRNA pathway due to the excessively high expression of shRNA from PolIII promoters (Fowler et al., 2016). Moreover, expression of both a therapeutic gene and a shRNA by a single vector has been typically achieved through employing a polymerase II (PolII) promoter driving the therapeutic gene and a PolIII promoter driving the shRNA of interest. This is functional, but comes at the cost of vector space and thus offers less options for including therapeutic genes (Chumakov et al., 2010; Moore et al., 2010).

Embedding the shRNA within a microRNA (mir) framework allows the shRNA to be processed under the control of a PolII promoter (Giering et al., 2008). Importantly, the level of expression of an embedded shRNA tends to be lower, thereby avoiding the toxicity observed expressed when using other systems, such as the U6 promoter (Fowler et al., 2015). Indeed, mice receiving a shRNA driven by a liver-specific PolII promoter showed stable gene knockdown with no tolerability issue for more than one year (Giering et al., 2008). However, this was only for one shRNA, done in liver cells, and the reduction at protein level was only 15% (Giering et al., 2008), so it is not known whether higher efficiency can be achieved, also for more than one target, and particularly in immune cells (which are harder to manipulate).

Surprisingly, starting from the observation that elements of the miR106a˜363 cluster, a paralog of the miR-17-92 cluster, are surprisingly efficient at downregulation of targets, and particularly multiplexed downregulation of targets in T cells, it is shown herein that multiplexed downregulation can further be improved by making chimeric clusters based on scaffolds of the miR-17 family cluster. This can be done in particular by making use of chimeric scaffolds that incorporate upper stem and loop regions of the miR-17 family on a lower stem region of a different scaffold from the miR-17 family cluster, and/or by combining scaffolds from different miR-17 paralogs; in particular scaffolds from the miR-17 family from different paralog clusters. The expression of multiple microRNA-based shRNAs (based on the individual scaffolds occurring e.g. in the miR106a˜363 cluster) against different targets was feasible in T cells without showing recombination, without showing toxicity and while simultaneously achieving efficient downregulation of multiple targets.

Accordingly, it is an object of the invention to provide specific nucleic acid molecules comprising miRNA scaffolds, vectors and engineered cells containing such nucleic acid molecules. The nucleic acid molecules, vectors and cells are typically provided for use as a medicament, such as use in the treatment of cancer. This is equivalent as saying that methods for treating cancer are provided, entailing the administration of nucleic acid molecules, vectors or cells as described herein, to a subject in need thereof, thereby improving at least one symptom of the cancer.

According to a first aspect, nucleic acid molecules are provided that contain at least one RNA interference molecule with an engineered scaffold, wherein the lower stem region of the scaffold is that of a miR scaffold from the miR-17 family cluster, and wherein at least part of the upper stem/loop region of the scaffold has been engineered to differ from the wild-type/natural sequence.

The miR-17 family cluster contains fifteen scaffolds, so the lower stem region of the engineered scaffold will be selected from a miR-17 scaffold, a miR-18a scaffold, a miR-19a scaffold, a miR-20a scaffold, a miR-19b-1 scaffold, a miR-92-1 scaffold, a miR-106a scaffold, a miR-18b scaffold, a miR-20b scaffold, a miR-19b-2 scaffold, a miR-92-2 scaffold, a miR-363 scaffold, a miR-106b scaffold, a miR-25 scaffold, and a miR-93 scaffold.

When at least part of the upper stem/loop region has been engineered to differ from the wild-type/natural sequence, this can mean that only the loop region has been engineered to this effect, only the upper stem region has been engineered to this effect, part of either or both the upper stem region and the loop have been engineered to this effect, or all of the upper stem and loop region have been engineered.

According to particular embodiments, the engineered scaffolds disclosed herein are chimeric scaffolds. Typically, they are chimeric scaffolds derived from two scaffolds from the miR-17 family cluster. Particularly, at least one of the two scaffolds will also be a miR-17 family scaffold, i.e., one selected from a miR-17 scaffold, a miR-20a scaffold, a miR-106a scaffold, a miR-20b scaffold, a miR-106b scaffold, and a miR-93 scaffold. Most particularly, the chimeric scaffold will contain a lower stem region selected from a miR-17 family cluster scaffold (i.e., selected from a miR-17 scaffold, a miR-18a scaffold, a miR-19a scaffold, a miR-20a scaffold, a miR-19b-1 scaffold, a miR-92-1 scaffold, a miR-106a scaffold, a miR-18b scaffold, a miR-20b scaffold, a miR-19b-2 scaffold, a miR-92-2 scaffold, a miR-363 scaffold, a miR-106b scaffold, a miR-25 scaffold, and a miR-93 scaffold) and at least part of the upper stem/loop region, but particularly all of the upper stem/loop region that is selected from a miR-17 family scaffold (i.e. selected from a miR-17 scaffold, a miR-20a scaffold, a miR-106a scaffold, a miR-20b scaffold, a miR-106b scaffold, and a miR-93 scaffold).

Accordingly, in a further embodiment, nucleic acid molecules are provided that contain at least one RNA interference molecule with an engineered scaffold, wherein the lower stem region of the scaffold is that of a miR scaffold selected from a miR-17 scaffold, a miR-18a scaffold, a miR-19a scaffold, a miR-20a scaffold, a miR-19b-1 scaffold, a miR-92-1 scaffold, a miR-106a scaffold, a miR-18b scaffold, a miR-20b scaffold, a miR-19b-2 scaffold, a miR-92-2 scaffold, a miR-363 scaffold, a miR-106b scaffold, a miR-25 scaffold, and a miR-93 scaffold, and wherein the upper stem and loop region of the scaffold are selected from a miR-17 scaffold, a miR-20a scaffold, a miR-106a scaffold, a miR-20b scaffold, a miR-106b scaffold, and a miR-93 scaffold; and wherein the scaffold from the lower stem region is different from the scaffold from the upper stem and loop region.

In addition to the scaffold, the at least one RNA interference molecule typically will also contain a target sequence not present in the wild-type/natural scaffold sequence. Particularly, the target sequence has a length of between 18-23 nucleic acids, more particularly a length of between 18-21 nucleic acids, most particularly a length of between 18 and 20 nucleic acids.

According to further particular embodiments, the nucleic acid molecules that contain at least one RNA interference molecule will contain at least two multiplexed RNA interference molecules, at least one of which has a scaffold as described above. When at least two multiplexed RNA interference molecules are present, those two or more molecules can have identical or different scaffolds. Although in principle, the additional RNA interference molecules can have any type of suitable scaffold, be it wild-type/natural or synthetic, it is particularly envisaged that the additional RNA interference molecule (or molecules) have a scaffold selected from the miR-17 family cluster, i.e. a scaffold selected from a miR-17 scaffold, a miR-18a scaffold, a miR-19a scaffold, a miR-20a scaffold, a miR-19b-1 scaffold, a miR-92-1 scaffold, a miR-106a scaffold, a miR-18b scaffold, a miR-20b scaffold, a miR-19b-2 scaffold, a miR-92-2 scaffold, a miR-363 scaffold, a miR-106b scaffold, a miR-25 scaffold, and a miR-93 scaffold. As shown in the Examples, in particular Examples 5 to 8, the combination of such scaffolds can lead to successful multiplexing. Importantly, not all of the scaffolds need to have an upper stem/loop region that is engineered to differ from the wild-type/natural sequence. According to particular embodiments, however, when at least two multiplexed RNA interference molecules are present, all of the scaffolds will have a lower stem region from a miR-17 family cluster.

As mentioned, the scaffolds can be identical or different. However, it is particularly envisaged that no more than three of the scaffolds are identical, and even more particularly envisaged that no more than two identical scaffolds are used. This to avoid recombination between identical scaffold sequences, or other factors reducing the miRNA processing (see Example 5).

Thus, according to a further aspect, nucleic acid molecules are provided containing at least two RNA interference molecules with different scaffolds, wherein the at least two different scaffolds have a lower stem region of a miR scaffold from the miR-17 family cluster; and wherein
at least one RNA interference molecule has a chimeric scaffold wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold; and/or wherein
the scaffolds from the at least two RNA interference molecules are miR-17 family scaffolds from at least two different miR-17 family clusters, i.e. selected from at least two different groups consisting of miR-17 and miR-20a (from the miR-17-92 cluster), miR-106a and miR-20b (from the 106a-363 cluster), and miR-106b and miR-93 (from the miR-106b-25 cluster).

Again, it is particularly envisaged that the at least part of the upper stem/loop region is the whole upper stem/loop region.

According to specific embodiments, the scaffolds present in the nucleic acid molecule are exclusively selected from the miR-17 family cluster (optionally with further engineering). However, it is also envisaged that these are further combined with different scaffold sequences, particularly different unrelated sequences (to avoid recombination), such as the miR-196a2 sequence and/or the miR-23a˜27a˜24-2 cluster.

According to further particular embodiments, the nucleic acid molecules that contain at least one RNA interference molecule will contain at least two multiplexed RNA interference molecules under control of one promoter. According to even further particular embodiments, the at least two multiplexed RNA interference molecules are at least three multiplexed RNA interference molecules. According to yet further embodiments, the at least two multiplexed RNA interference molecules are at least four multiplexed RNA interference molecules; the at least two multiplexed RNA interference molecules are at least five multiplexed RNA interference molecules; the at least two multiplexed RNA interference molecules are at least six multiplexed RNA interference molecules.

According to particular embodiments, a scaffold sequence may have been engineered to reduce the number of mismatches and/or bulges in the stem region. A “mismatch” as used herein refers to a base pair that is not a complimentary Watson-Crick base pair. A “bulge” as used herein refers to an unpaired stretch of nucleotides (typically 1-5, particularly 1-3) located within one strand of a nucleic acid duplex. More particularly, if one of the scaffold sequences that is used is a miR-18b scaffold, the scaffold can have been engineered (and is modified compared to the wild-type/natural sequence) to reduce the number of mismatches and/or bulges in the stem region (see Example 3). This can be done by restoring base pair complementarity (in case of a mismatch), typically by matching the passenger strand to the target strand, or by removing the superfluous unpaired nucleotides in case of a bulge.

According to particular embodiments, the at least two multiplexed RNA interference molecules can be shRNA molecules or miRNA molecules. Most particularly, they are miRNA molecules. A difference between shRNA molecules and miRNA molecules is that miRNA molecules are processed by Drosha, while conventional shRNA molecules are not (which has been associated with toxicity, Grimm et al., Nature 441:537-541 (2006)).

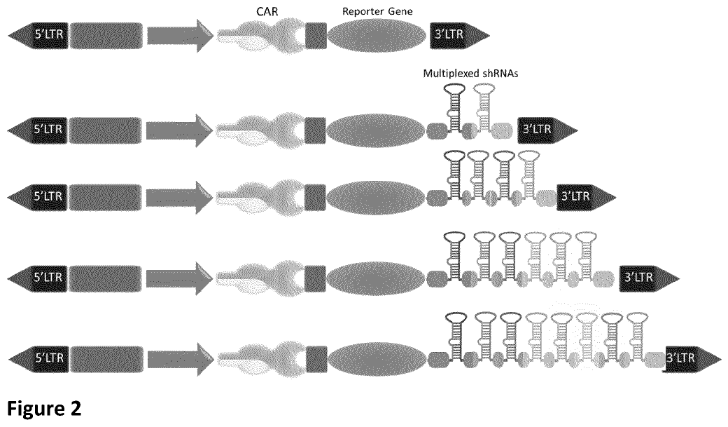

According to specific embodiments, the miRNA molecules can be provided as individual miRNA scaffolds under control of one promoter. Each scaffold selected normally corresponds to one miRNA (FIG. 1), the scaffold can be repeated or combined with other scaffolds to obtain the expression of multiple RNA interference molecules (FIG. 1-2). However, when repeating or combining with further scaffolds, it is typically envisaged that all of the multiplexed RNA interference molecules will be under control of one promoter (i.e., the promoter is not repeated when the individual scaffold is repeated, or another scaffold is added).

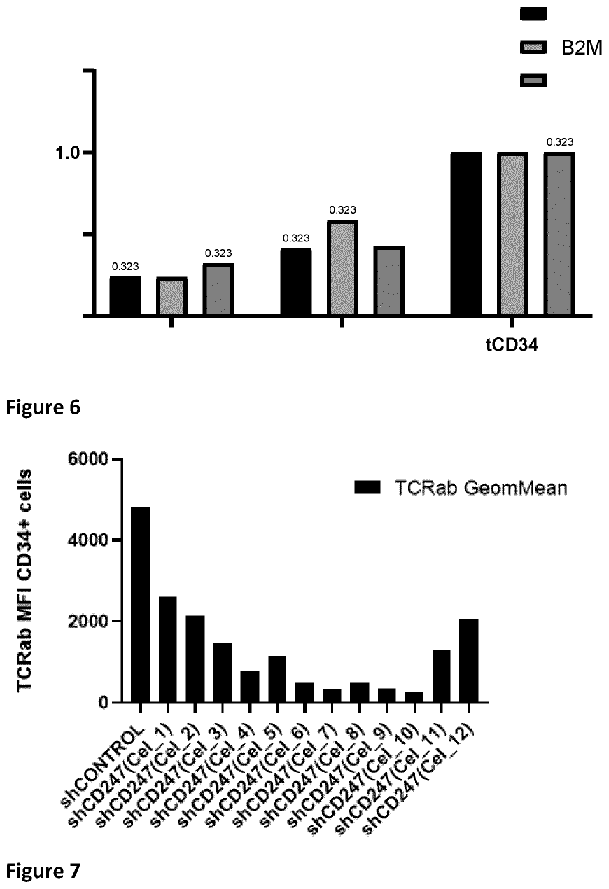

Particularly suited scaffold sequences for miRNA multiplexing are those found in authentic polycistronic miRNA clusters or parts thereof, where the endogenous miRNA target sequence is replaced by a shRNA target sequence of interest. Particularly suitable miR scaffold clusters to this end are the miR-106a˜363, miR-17˜92, miR-106b˜25, and miR-23a˜27a˜24-2 cluster; most particularly envisaged is the miR-106a˜363 cluster and fragments (i.e. one or more individual scaffolds) thereof. Of note, to save vector payload, it is also specifically envisaged to use part of such wild-type/natural clusters and not the whole sequence (this is particularly useful as not all miRNAs are equally interspaced, and not all linker sequences may be needed). Indeed, it is shown herein (Example 5) that scaffolds can be used outside of the cluster context and be combined in different ways. Other considerations can be taken into account, e.g. taking the miRNAs that are most efficiently processed in a cell. For instance, the miR-17˜92 cluster consists of (in order) the miR-17 scaffold, the miR-18a scaffold, the miR-19a scaffold, the miR-20a scaffold, the miR-19b-1 scaffold and the miR-92-1 (also miR-92a1) scaffold, particularly useful fragments of the cluster are the scaffold sequence from miR-19a to miR-92-1 (i.e. 4 of the 6 miRNAs) with their linkers, or from miR-19a to miR-19b-1 (3 of the 6 miRNAs). Likewise, the 106a˜363 cluster consists of (in order) the miR-106a scaffold, the miR-18b scaffold, the miR-20b scaffold, the miR-19b-2 scaffold, the miR-92-2 (also miR-92a2) scaffold and the miR-363 scaffold (see FIG. 5). Particularly useful fragments of the cluster are the scaffold sequences from miR-106a to miR-20b (i.e. 3 of the 6 miRNAs) (see Example 5), miR-20b to miR-363 (i.e. 4 of the 6 miRNAs) or from miR-19b-2 to miR-363 (i.e. 3 of the 6 miRNAs) (see FIG. 6). Both the wild-type/natural linker sequences can be used, as well as fragments thereof or artificial linkers (again to reduce payload of the vectors).

As miRNA scaffolds from the miR-106a˜363 cluster are particularly envisaged, particularly envisaged linkers are the sequences 5′ and 3′ of the respective scaffold (see FIG. 1). Linker sequences can e.g. be 150 bp, 140 bp, 130 bp, 120 bp, 110 bp, 100 bp, 90 bp, 80 bp, 70 bp, 60 bp, 50 bp, 40 bp, 30 bp, 20 bp, 10 bp or less on either side of the scaffold. When two scaffolds are used that are non-adjacent in the cluster (as e.g. in Example 5), the linkers are by definition not identical as those found in the clusters. Still, one could use e.g. 30, 60 or 90 bp present 3′ of one scaffold in the cluster and fuse it to a linker consisting of 30, 60, 90 bp 5′ of the next selected scaffold, creating a hybrid linker.

The miRNA scaffolds are particularly used as such: i.e., without modification to the scaffold sequence. Particularly the lower stem sequence will be kept identical to that found in the respective miRNA scaffold. Preferably, the loop sequences in the upper stem are not changed either, but experiments have shown that these are primarily flexible structures, and length and sequence can be adapted as long as the upper stem structure is not affected. Although not preferred, the skilled person will appreciate that scaffolds with such modified loops are within the scope of this application. Within the upper stem of the scaffolds, the target sequence is found. Wild-type/natural target sequences of the miR-106a-363 cluster are 22 to 23 bp long. As shown in Example 4, target sequences can be shortened in size without deleterious effects. Target sequences can be from 18 to 23 bp long, and sequences from 18 to 21 bp are particularly envisaged; sequences from 18 to 20 bp are even more particularly envisaged. When shorter sequences are needed, it is no problem to use target sequences of 18 or 19 bp.

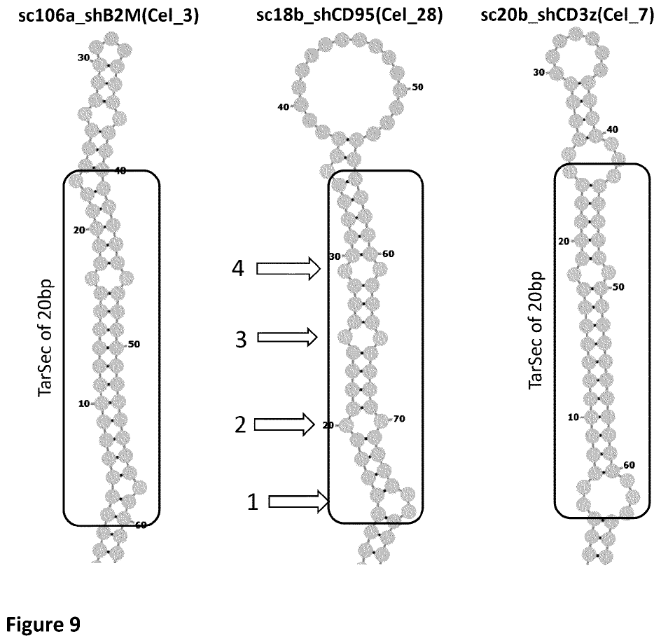

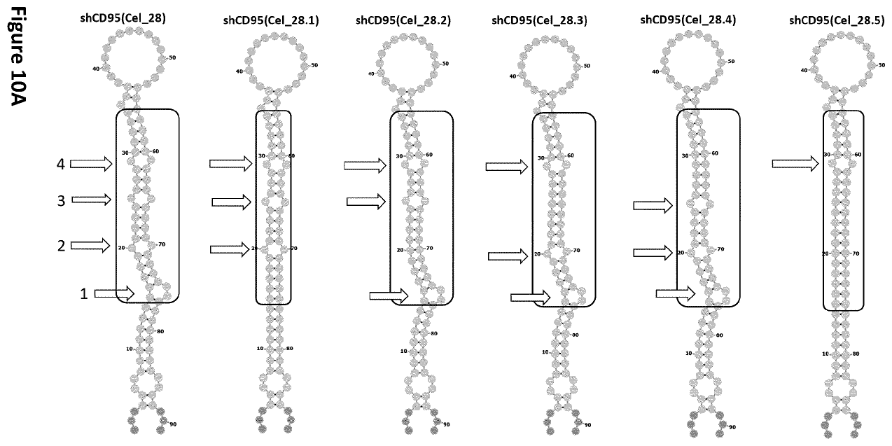

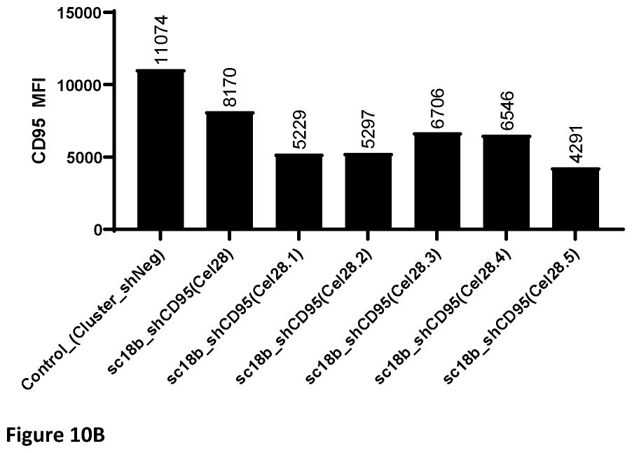

As is evident for sake of targeting, the target sequence is the part of the scaffold that obviously requires adaptation to the target. As the miRNA scaffolds have some mismatches in their architecture, question is whether these mismatches should be retained. As shown in Example 3 (and FIG. 9), the mismatch found at position 14 of the target sequence in miR-106a and miR-20b can be retained without any negative effect on downregulation of the target, meaning that the passenger strand is not perfectly complimentary to the guide strand. As also shown in Example 3 (and FIG. 10), when more than one mismatch is present (such as in the miR-18b scaffold), the passenger strand can be made more complimentary to the guide strand to achieve a more efficient knockdown (when needed). Note that this modification is not needed to achieve significant levels of knockdown, but eliminating mismatches at position 6, 11 and 15 of the target sequence (corresponding to bp 20 and 70, 25 and 65 and 29 and 61 of the scaffold (see FIG. 9)) does systematically improve knockdown. The same can be said for the bulge (nucleotides 75 and 76 of the miR-18b scaffold). Increasing complementarity of target and passenger strand by removing mismatches or bulges in the passenger strand likely improves the downregulation in other scaffolds as well, although this has not yet been needed, as testing different target sequences always yielded satisfactory knockdown levels.

Each RNA interference molecule can target a different molecule, they can target the same molecule, or a combination thereof (i.e. more than one RNA molecule directed against one target, while only one RNA interference molecule is directed against a different target). When the RNA interference molecules are directed against the same target, they can target the same region, or they can target a different region. In other words, the RNA interference molecules can be identical or not when directed against the same target. Examples of such combinations of RNA interference molecules are shown in the Examples section.

Thus, according to particular embodiments, at least two of the multiplexed RNA interference molecules are directed against the same target. According to further particular embodiments, these at least two RNA interference molecules use identical miRNA scaffolds. They can be directed against the same target by using the same target sequence (according to these specific embodiments, at least two of the multiplexed RNA interference molecules are identical) or by using a different target sequence (according to these specific embodiments, at least two of the multiplexed RNA interference molecules have identical scaffolds, but differing target sequence). According to alternative embodiments, the at least two multiplexed RNA interference molecules directed against the same target have a different miRNA scaffold sequence. In that case, they can have the same target sequence, or can have a different target sequence directed against the same target.

According to alternative embodiments, all of the at least two multiplexed RNA interference molecules are different. According to further specific embodiments, all of the at least two multiplexed RNA interference molecules are directed against different targets.

Any suitable molecule present in the engineered cell can be targeted by the instant RNA interference molecules. Typical examples of envisaged targets are: a MHC class I gene, a MHC class II gene, a MHC coreceptor gene (e.g. HLA-F, HLA-G), a TCR chain, NKBBIL, LTA, TNF, LTB, LST1, NCR3, AIF1, LY6, a heat shock protein (e.g. HSPA1L, HSPA1A, HSPA1B), complement cascade, regulatory receptors (e.g. NOTCH4), TAP, HLA-DM, HLA-DO, RING1, CD52, CD247, HCP5, B2M, MICA, MICB, ULBP1, ULBP2, ULBP3, ULBP4, ULBP5, ULBP6, 2B4, A2AR, BAX, BLIMP1, C160 (POLR3A), CBL-B, CCR6, CD7, CD27, CD28, CD38, CD95, CD96, CD123, CD272 (BTLA), CD276 (aka B7-H3), CIITA, CTLA4, DGK [DGKA, DGKB, DGKD, DGKE, DKGG, DGKH, DGKI, DGKK, DGKQ, DGKZ], DNMT3A, DR4, DR5, EGR2, FABP4, FABP5, FASN, GMCSF, HPK1, IL-10R [IL10RA, IL10RB], IL2, LAG3 (CD223), LFA1, NEAT 1, NFkB (including RELA, RELB, NFkB2, NFkB1, REL), NKG2A, NR4A (including NR4A1, NR4A2, NR4A3), PD1, PI3KCD, PPP2RD2, PRAS40, RAPTOR, SHIP1, SOAT1, SOCS1, T-BET, TCF7 (aka TCF-1), TET2, TGFBR1, TGFBR2, TGFBR3, TIGIT, TIM3 (aka HAVCR2 or CD366), TOX, TOX2, VISTA (aka VSIR or B7-H5), ZC3H12A (also known as regnase-1 or MCPIP) and ZFP36L2.

According to a further aspect of the invention, the nucleic acid molecules are not used as such, but provided in a suitable vector, i.e. a vector that allows expression in cells. According to particular embodiments, the vectors are suitable for expression in eukaryotic cells, particularly in immune cells.

Thus, vectors that are suitable for expression in engineered immune cells are provided that comprise a nucleic acid molecule as described herein. All of the features disclosed for the nucleic acid molecules apply to the vectors mutatis mutandis. In other words, vectors are provided that comprise at least one RNA interference molecule with an engineered scaffold, wherein the lower stem region of the scaffold is that of a miR scaffold from the miR-17 family cluster, and wherein at least part of the upper stem/loop region of the scaffold has been engineered to differ from the natural sequence. According to further embodiments, the lower stem region of the engineered scaffold is selected from a miR-17 scaffold, a miR-18a scaffold, a miR-19a scaffold, a miR-20a scaffold, a miR-19b-1 scaffold, a miR-92-1 scaffold, a miR-106a scaffold, a miR-18b scaffold, a miR-20b scaffold, a miR-19b-2 scaffold, a miR-92-2 scaffold, a miR-363 scaffold, a miR-106b scaffold, a miR-25 scaffold, and a miR-93 scaffold. According to further embodiments, the engineered scaffold is a chimeric scaffold and wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold.

According to particular embodiments, the at least one RNA interference molecule present in the vector are at least two RNA interference molecules, particularly at least two multiplexed RNA interference molecules.

According to further particular embodiments, provided are vectors containing a nucleic acid molecule comprising at least two RNA interference molecules with different scaffolds, wherein the at least two different scaffolds have a lower stem region of a miR scaffold from the miR-17 family cluster; and wherein
at least one RNA interference molecule has a chimeric scaffold wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold; and/or wherein
the scaffolds from the at least two RNA interference molecules are a miR-17 family scaffold from different miR-17 family clusters.

The at least two multiplexed RNA interference molecules can be at least three, at least four, at least five, at least six, at least seven, at least eight, at least nine, at least ten or even more molecules, depending on the number of target molecules to be downregulated and practical considerations in terms of co-expressing the multiplexed molecules. The miR-17 family cluster has fifteen scaffolds, scaffolds can be duplicated without loss of knockdown activity (Example 5), and individual scaffolds from the different clusters can be combined (Example 7), so up to 12 scaffolds can in principle be multiplexed, although in practice often a lower number will be used.

A “multiplex” is a polynucleotide that encodes for a plurality of molecules of the same type, e.g., a plurality of siRNA or shRNA or miRNA. Within a multiplex, when molecules are of the same type (e.g., all shRNAs), they may be identical or comprise different sequences. Between molecules that are of the same type, there may be intervening sequences such as linkers, as described herein. An example of a multiplex of the present invention is a polynucleotide that encodes for a plurality of tandem miRNA-based shRNAs. A multiplex may be single stranded, double stranded or have both regions that are single stranded and regions that are double stranded.

According to particular embodiments, the at least two multiplexed RNA interference molecules are under control of one promoter. Typically, when more than one RNA interference molecule is expressed, this is done by incorporating multiple copies of a shRNA-expression cassette. These typically carry identical promoter sequences, which results in frequent recombination events that remove the repeated sequence fragments. As a solution, typically several different promoters are used in an expression cassette (e.g. Chumakov et al., 2010). According to the present embodiments, however, recombination is avoided by the use of only one promoter. While expression is typically lower, this has advantages in terms of toxicity, as too much siRNA can be toxic to the cell (e.g. by interfering with the endogenous siRNA pathway). The use of only one promoter has the added advantage that all shRNAs are coregulated and expressed at similar levels. Remarkably, as shown in the Examples, multiple shRNAs can be transcribed from one promoter without a significant drop in efficacy.

Typically, the promoter used to express the RNA interference molecules is not a U6 promoter. This because this promoter is linked to toxicity, particularly at high levels of expression. For the same reason, one can consider to exclude H1 promoters (which are weaker promoters than U6) or even Pol III promoters in general (although they can be suitable in certain conditions). Thus, according to specific embodiments, the promoter used to express the RNA interference molecules is not a RNA Pol III promoter. RNA Pol III promoters lack temporal and spatial control and do not allow controlled expression of miRNA inhibitors. In contrast, numerous RNA Pol II promoters allow tissue-specific expression, and both inducible and repressible RNA Pol II promoters exist. Although tissue-specific expression is often not required in the context of the invention (as cells are selected prior to engineering), having specific promoters for e.g. immune cells is still an advantage, as it has been shown that differences in RNAi efficacy from various promoters were particularly pronounced in immune cells (Lebbink et al., 2011). According to specific embodiments, the promoter is selected from a Pol II promoter, and a Pol III promoter. According to particular embodiments, the promoter is a natural or synthetic Pol II promoter. Suitable promoters include, but are not limited to, a cytomegalovirus (CMV) promoter, an elongation factor 1 alpha (EF1α) promoter (core or full length), a phosphoglycerate kinase (PGK) promoter, a composite beta-actin promoter with an upstream CMV IV enhancer (CAG promoter), a ubiquitin C (UbC) promoter, a spleen focus forming virus (SFFV) promoter, a Rous sarcoma virus (RSV) promoter, an interleukin-2 promoter, a murine stem cell virus (MSCV) long terminal repeat (LTR), a Gibbon ape leukemia virus (GALV) LTR, a simian virus 40 (SV40) promoter, and a tRNA promoter. These promoters are among the most commonly used polymerase II promoters to drive mRNA expression.

The vectors disclosed herein are particularly suitable for use in cells used for ACT. Accordingly, it is an object of the invention to provide engineered cells comprising a nucleic acid molecule encoding at least one RNA interference molecule as described herein. The RNA interference molecules typically also contain a target sequence not present in the natural scaffold sequence. The target sequence typically has a length of between 18-23 nucleic acids. It is particularly envisaged that the target sequence is directed against a sequence occurring in the engineered cells, particularly a sequence of a target. I.e., the at least one RNA interference molecule has a sequence targeting (by means of base pair complementarity) a sequence in the engineered cell encoding a protein to be downregulated, or regulatory regions of the target protein. Examples of such targets include, but are not limited to, a MHC class I gene, a MHC class II gene, a MHC coreceptor gene (e.g. HLA-F, HLA-G), a TCR chain, NKBBIL, LTA, TNF, LTB, LST1, NCR3, AIF1, LY6, a heat shock protein (e.g. HSPA1L, HSPA1A, HSPA1B), complement cascade, regulatory receptors (e.g. NOTCH4), TAP, HLA-DM, HLA-DO, RING1, CD52, CD247, HCP5, B2M, MICA, MICB, ULBP1, ULBP2, ULBP3, ULBP4, ULBP5, ULBP6, 2B4, A2AR, BAX, BLIMP1, C160 (POLR3A), CBL-B, CCR6, CD7, CD27, CD28, CD38, CD95, CD96, CD123, CD272 (BTLA), CD276 (aka B7-H3), CIITA, CTLA4, DGK [DGKA, DGKB, DGKD, DGKE, DKGG, DGKH, DGKI, DGKK, DGKQ, DGKZ], DNMT3A, DR4, DR5, EGR2, FABP4, FABP5, FASN, GMCSF, HPK1, IL-10R [IL10RA, IL10RB], IL2, LAG3 (CD223), LFA1, NEAT 1, NFkB (including RELA, RELB, NFkB2, NFkB1, REL), NKG2A, NR4A (including NR4A1, NR4A2, NR4A3), PD1, PI3KCD, PPP2RD2, PRAS40, RAPTOR, SHIP1, SOAT1, SOCS1, T-BET, TCF7 (aka TCF-1), TET2, TGFBR1, TGFBR2, TGFBR3, TIGIT, TIM3 (aka HAVCR2 or CD366), TOX, TOX2, VISTA (aka VSIR or B7-H5), ZC3H12A (also known as regnase-1 or MCPIP) and ZFP36L2.

In addition to the RNA interference molecule, the vector will often contain further elements, and typically also contains nucleic acid encoding a protein of interest, such as a CAR. According to further particular embodiments, both the at least two multiplexed RNA interference molecules and the protein of interest are under control of one promoter. This again reduces vector load (as no separate promoter is used to express the protein of interest), and offers the advantage of coregulated expression. This can e.g. be advantageous when the protein of interest is a CAR that targets a cancer, and the RNA interference molecules are intended to have an added or synergistic effect in tumor eradication. Examples of useful RNA targets include (without limitation) CD247, TRAC (both downregulating the TCR complex, making the cells more suitable for allogeneic therapy), B2M (to expand histocompatibility), CD52 (making the cells survive CD52-directed chemotherapy), CD95 (making the cells insensitive to CD95-induced cell death), checkpoint molecules (e.g. PD-1, PD-L1, CTLA4), and many more.

As mentioned, the nucleic acid molecules and vectors described herein are particularly useful for engineering cells for ACT. Thus, engineered immune cells are provided that comprise a nucleic acid molecule or a vector as described herein. All of the features disclosed for the nucleic acid molecules and vectors apply to the engineered cells mutatis mutandis.

Cells containing at least one RNA interference molecule, or containing at least two RNA interference molecules, can have advantages, particularly therapeutic benefits. RNA interference molecules can indeed be directed against targets of which (over) expression is undesirable. However, typically, the engineered cells provided herein will further contain at least one protein of interest.

Accordingly, engineered cells are provided that contain
a first exogenous nucleic acid molecule encoding a protein of interest, and
a second nucleic acid molecule comprising at least one RNA interference molecule with an engineered scaffold, wherein the lower stem region of the scaffold is that of a miR scaffold from the miR-17 family cluster, and wherein at least part of the upper stem/loop region of the scaffold has been engineered to differ from the natural sequence.

According to further embodiments, the lower stem region of the engineered scaffold is selected from a miR-17 scaffold, a miR-18a scaffold, a miR-19a scaffold, a miR-20a scaffold, a miR-19b-1 scaffold, a miR-92-1 scaffold, a miR-106a scaffold, a miR-18b scaffold, a miR-20b scaffold, a miR-19b-2 scaffold, a miR-92-2 scaffold, a miR-363 scaffold, a miR-106b scaffold, a miR-25 scaffold, and a miR-93 scaffold. According to further embodiments, the engineered scaffold is a chimeric scaffold and wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold.

Additionally, engineered cells are provided comprising:
a first exogenous nucleic acid molecule encoding a protein of interest, and
a second nucleic acid molecule comprising at least two RNA interference molecules with different scaffolds, wherein the at least two different scaffolds have a lower stem region of a miR scaffold from the miR-17 family cluster; and wherein
   at least one RNA interference molecule has a chimeric scaffold wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold; and/or wherein
   the scaffolds from the at least two RNA interference molecules are a miR-17 family scaffold from different miR-17 family clusters.

According to particular embodiments, the engineered cells are engineered immune cells. Particularly, the immune cells are selected from a T cell, a NK cell, a NKT cell, a macrophage, a stem cell, a progenitor cell, and an iPSC cell.

When at least two multiplexed RNA interference molecules are present, those two or more molecules can have identical or different scaffolds. However, it is particularly envisaged that no more than three of the scaffolds are identical, and even more particularly envisaged that no more than two identical scaffolds are used. This to avoid recombination between identical scaffold sequences, or overload of the miRNA processing capacity of the cell (see Example 5). For the same reason, when there is more than one target sequence directed to the same target, it is particularly envisaged that either a different target sequence is used, or that the identical target sequence is used in a different scaffold. Identical target sequences in identical scaffolds are possible, but it is particularly envisaged that they occur not more than twice.

The optional further additional protein of interest can e.g. provide an additive, supportive or even synergistic effect, or it can be used for a different purpose. For instance, the protein of interest can be a CAR directed against a tumor, and the RNA interference molecules may interfere with tumor function, e.g. by targeting an immune checkpoint, directly downregulating a tumor target, targeting the tumor microenvironment. Alternatively or additionally, one or more of the RNA interference molecules may prolong persistence of the therapeutic cells, or otherwise alter a physiological response (e.g. interfering with GvHD or host versus graft reaction).

Proteins of interest can in principle be any protein, depending on the setting. However, typically they are proteins with a therapeutic function. These may include secreted therapeutic proteins, such as e.g. interleukins, cytokines or hormones. However, according to particular embodiments, the protein of interest is not secreted. Instead of a therapeutic protein, the protein of interest can serve a different function, e.g. diagnostic, or detection. Thus, the protein of interest can be a tag or reporter gene. Typically, the protein of interest is a receptor. According to further particular embodiments, the receptor is a chimeric antigen receptor or a TCR. Chimeric antigen receptors can be directed against any target expressed on the surface of a target cell, typical examples include, but are not limited to, CD5, CD19, CD20, CD22, CD23, CD30, CD33, CD38, CD44, CD56, CD70, CD123, CD133, CD138, CD171, CD174, CD248, CD274, CD276, CD279, CD319, CD326, CD340, BCMA, B7H3, B7H6, CEACAM5, EGFRvIII, EPHA2, mesothelin, NKG2D, HER2, HER3, GPC3, Flt3, DLL3, IL1RAP, KDR, MET, mucin 1, IL13Ra2, FOLH1, FAP, CA9, FOLR1, ROR1, GD2, PSCA, GPNMB, CSPG4, ULBP1, ULBP2, but many more exist and are also suitable. Although most CARs are scFv-based (i.e., the binding moiety is a scFv directed against a specific target, and the CAR is typically named after the target), some CARs are receptor-based (i.e., the binding moiety is part of a receptor, and the CAR typically is named after the receptor). An example of the latter is an NKG2D-CAR.

Engineered TCRs can be directed against any target of a cell, including intracellular targets. In addition to the above listed targets present on a cell surface, typical targets for a TCR include, but are not limited to, NY-ESO-1, PRAME, AFP, MAGE-A3, MAGE-A4, MAGE-A6, MAGE-A10, MAGE-A12, gp100, MART-1, tyrosinase, WT1, p53, HPV-E6, HPV-E7, HBV, TRAIL, thyroglobulin, KRAS, HERV-E, HA-1, CMV, and CEA.

According to these particular embodiments where a further protein of interest is present, the first and second nucleic acid molecule in the engineered cell are typically present in one vector, such as a eukaryotic expression plasmid, a mini-circle DNA, or a viral vector (e.g. derived from a lentivirus, a retrovirus, an adenovirus, an adeno-associated virus, and a Sendai virus). According to further specific embodiments, the viral vector is selected from a lentiviral vector and a retroviral vector. Particularly for the latter vector load (i.e. total size of the construct) is important and the use of compact multiplex cassettes is particularly advantageous.

Of note, the cells described herein may contain more than one protein of interest: for instance a receptor protein and a reporter protein (see FIG. 2). Or a receptor protein, an interleukin and a tag protein.

The engineered cells are particularly eukaryotic cells, more particularly engineered mammalian cells, more particularly engineered human cells. According to particular embodiments, the cells are engineered immune cells. Typical immune cells are selected from a T cell, a NK cell, a NKT cell, a macrophage, a stem cell, a progenitor cell, and an iPSC cell.

The cells disclosed herein typically contain multiplexed RNA interference molecules. These can be directed against one or more targets which need to be downregulated (either targets within the cell, or outside of the cell if the shRNA is secreted).

An alternative way of phrasing the invention disclosed herein is that particularly suitable constructs have been identified which are miRNA-based. Accordingly, provided are engineered cells comprising a polynucleotide comprising a microRNA-based shRNA encoding region, wherein said microRNA-based shRNA encoding region comprises sequences that encode:

One or more artificial miRNA-based shRNA nucleotide sequences, wherein each artificial miRNA-based shRNA nucleotide sequence comprises
a miRNA scaffold sequence,
an active or mature sequence, and
a passenger or star sequence, wherein within each artificial miRNA-based shRNA nucleotide sequence, the active sequence is at least 70% complementary to the passenger sequence.

According to particular embodiments, the active sequence is at least 80% complementary to the passenger sequence, and can be at least 90% complementary to the passenger sequence or more.

A particular advantage is that the instant miRNA-based shRNA nucleotide sequences can be multiplexed. Accordingly, provided are engineered cells comprising a polynucleotide comprising a multiplexed microRNA-based shRNA encoding region, wherein said multiplexed microRNA-based shRNA encoding region comprises sequences that encode:

Two or more artificial miRNA-based shRNA nucleotide sequences, wherein each artificial miRNA-based shRNA nucleotide sequence comprises
a miRNA scaffold sequence,
an active or mature sequence, and
a passenger or star sequence, wherein within each artificial miRNA-based shRNA nucleotide sequence, the active sequence is at least 70% complementary to the passenger sequence.

The miRNA-based shRNA nucleotide sequences particularly are selected from a miR-106a sequence, a miR-18b sequence, a miR-20b sequence, a miR-19b-2 sequence, a miR-92-2 sequence and a miR-363 sequence. Both the active sequence and the passenger sequence of each of the artificial miRNA-based shRNA nucleotide sequences are typically between 18 and 40 nucleotides long, more particularly between 18 and 30 nucleotides, more particularly between 18 and 25 nucleotides, most particularly between 18 and 23 nucleotides long. The active sequence can also be 18 or 19 nucleotides long.

Typically, the passenger sequence has the same length as the active sequence, although the possible presence of bulges means that they are not always identical in length.

Typically, these microRNA scaffold sequences are separated by linkers. In microRNA clusters, linkers can be long: up to 500 nucleotides, up to 400 nucleotides, up to 300 nucleotides, up to 200 nucleotides, up to 150 nucleotides, up to 100 nucleotides. When multiplexing scaffold sequences, the objective can be to use natural linker sequences (those found 5′ and 3′ of the miRNA scaffold sequence) of sufficient length to ensure any potential regulatory sequence is included. For instance, one can use 50, 100 or 150 nucleotides flanking the scaffold sequence. An alternative objective can be to reduce vector payload and reduce linker length, and linker sequences can then e.g. be between 30 and 60 nucleotides long, although shorter stretches also work. In fact, it was surprisingly found that length of linker plays no vital role and can be very short (less than 10 nucleotides) or even be absent without interfering with shRNA function. According to particular embodiments, at least some of the 5′ and/or 3′ linker sequence is used with its respective scaffold. At least some typically is at least 10 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 120 nucleotides, at least 150 nucleotides, or at least 200 nucleotides of the 5′ and/or 3′ linker sequence.

The miRNA-based shRNA nucleotide sequences are considered artificial sequences, because even though the scaffold sequence may be naturally occurring, the endogenous miR sequences have been replaced by shRNA sequences engineered against a particular target. Artificial sequences can e.g. be naturally occurring scaffolds (e.g. a miR cluster or fragment thereof, such as the miR-106a˜363 cluster) wherein the endogenous miR sequences have been replaced by shRNA sequences engineered against a particular target, can be repeats of a single miR scaffold (such as e.g. the miR-20b scaffold) wherein the endogenous miR sequences have been replaced by shRNA sequences engineered against a particular target, can be artificial miR-like sequences, or a combination thereof.

This engineered cell typically further comprises a nucleic acid molecule encoding a protein of interest, such as a chimeric antigen receptor or a TCR, and can be an engineered immune cell, as described above.

The expression of the at least one RNA interference molecule or co-expression of the multiplexed RNA interference molecules results in the suppression of at least one gene, but typically a plurality of genes, within the engineered cells. This can contribute to greater therapeutic efficacy.

The engineered cells described herein are also provided for use as a medicament. According to specific embodiments, the engineered cells are provided for use in the treatment of cancer. Exemplary types of cancer that can be treated include, but not limited to, adenocarcinoma, adrenocortical carcinoma, anal cancer, astrocytoma, bladder cancer, bone cancer, brain cancer, breast cancer, cervical cancer, colorectal cancer, endometrial cancer, esophageal cancer, Ewing sarcoma, eye cancer, Fallopian tube cancer, gastric cancer, glioblastoma, head and neck cancer, Kaposi sarcoma, kidney cancer, leukemia, liver cancer, lung cancer, lymphoma, melanoma, mesothelioma, myelodysplastic syndrome, multiple myeloma, neuroblastoma, osteosarcoma, ovarian cancer, pancreatic cancer, parathyroid cancer, penile cancer, peritoneal cancer, pharyngeal cancer, prostate cancer, renal cell carcinoma, retinoblastoma, rhabdomyosarcoma, sarcoma, skin cancer, small intestine cancer, stomach cancer, testicular cancer, thyroid cancer, urethral cancer, uterine cancer, vaginal cancer, and Wilms tumor.

According to particular embodiments, the cells can be provided for treatment of liquid or blood cancers. Examples of such cancers include e.g. leukemia (including a.o. acute myelogenous leukemia (AML), acute lymphocytic leukemia (ALL), chronic myelogenous leukemia (CML), and chronic lymphocytic leukemia (CLL)), lymphoma (including a.o. Hodgkin's lymphoma and non-Hodgkin's lymphoma such as B-cell lymphoma (e.g. DLBCL), T cell lymphoma, Burkitt's lymphoma, follicular lymphoma, mantle cell lymphoma, and small lymphocytic lymphoma), multiple myeloma or myelodysplastic syndrome (MDS).

This is equivalent as saying that methods of treating cancer are provided, comprising administering to a subject in need thereof a suitable dose of engineered cells as described herein (i.e. engineered cells comprising an exogenous nucleic acid molecule encoding at least two multiplexed RNA interference molecules, and optionally comprising a further nucleic acid molecule encoding a protein of interest), thereby improving at least one symptom associated with the cancer. Cancers envisaged for treatment include, but are not limited to, adenocarcinoma, adrenocortical carcinoma, anal cancer, astrocytoma, bladder cancer, bone cancer, brain cancer, breast cancer, cervical cancer, colorectal cancer, endometrial cancer, esophageal cancer, Ewing sarcoma, eye cancer, Fallopian tube cancer, gastric cancer, glioblastoma, head and neck cancer, Kaposi sarcoma, kidney cancer, leukemia, liver cancer, lung cancer, lymphoma, melanoma, mesothelioma, myelodysplastic syndrome, multiple myeloma, neuroblastoma, osteosarcoma, ovarian cancer, pancreatic cancer, parathyroid cancer, penile cancer, peritoneal cancer, pharyngeal cancer, prostate cancer, renal cell carcinoma, retinoblastoma, rhabdomyosarcoma, sarcoma, skin cancer, small intestine cancer, stomach cancer, testicular cancer, thyroid cancer, urethral cancer, uterine cancer, vaginal cancer, and Wilms tumor. According to further particular embodiments, methods of treating blood cancer are provided, comprising administering to a subject in need thereof a suitable dose of engineered cells as described herein thereby improving at least one symptom of the cancer.

According to alternative embodiments, the cells can be provided for use in the treatment of autoimmune disease. Exemplary types of autoimmune diseases that can be treated include, but are not limited to, rheumatoid arthritis (RA), systemic lupus erythematosus (SLE), inflammatory bowel disease (IBD), multiple sclerosis (MS), Type 1 diabetes mellitus, amyotrophic lateral sclerosis (ALS or Lou Gehrig's disease), spinal muscular atrophy (SMA), Crohn's disease, Guillain-Barre syndrome, chronic inflammatory demyelinating polyneuropathy, psoriasis, psoriatic arthritis, Addison's disease, ankylosing spondylitis, Behcet's disease, coeliac disease, Coxsackie myocarditis, endometriosis, fibromyalgia, Graves' disease, Hashimoto's thyroiditis, Kawasaki disease, Meniere's disease, myasthenia gravis, sarcoidosis, scleroderma, Sjögren's syndrome, thrombocytopenia purpura (TTP), ulcerative colitis, vasculitis and vitiligo.

This is equivalent as saying that methods of treating autoimmune disease are provided, comprising administering to a subject in need thereof a suitable dose of engineered cells as described herein, thereby improving at least one symptom associated with the autoimmune disease. Exemplary autoimmune diseases that can be treated are listed above.

According to yet further embodiments, the cells can be provided for use in the treatment of infectious disease. “Infectious disease” is used herein to refer to any type of disease caused by the presence of an external organism (pathogen) in or on the subject or organism with the disease. Infections are usually considered to be caused by microorganisms or microparasites like viruses, prions, bacteria, and viroids, though larger organisms like macroparasites and fungi can also infect. The organisms that can cause infection are herein referred to as “pathogens” (in case they cause disease) and “parasites” (in case they benefit at the expense of the host organism, thereby reducing biological fitness of the host organism, even without overt disease being present) and include, but are not limited to, viruses, bacteria, fungi, protists (e.g. Plasmodium, Phytophthora) and protozoa (e.g. Plasmodium, Entamoeba, Giardia, Toxoplasma, Cryptosporidium, Trichomonas, Leishmania, Trypanosoma) (microparasites) and macroparasites such as worms (e.g. nematodes like ascarids, filarias, hookworms, pinworms and whipworms or flatworms like tapeworms and flukes), but also ectoparasites such as ticks and mites. Parasitoids, i.e. parasitic organisms that sterilize or kill the host organism, are envisaged within the term parasites. According to particular embodiments, the infectious disease is caused by a microbial or viral organism.

“Microbial organism,” as used herein, may refer to bacteria, such as gram-positive bacteria (eg, Staphylococcus sp., Enterococcus sp., Bacillus sp.), Gram-negative bacteria. (for example, Escherichia sp., Yersinia sp.), spirochetes (for example, Treponema sp, such as Treponema pallidum, Leptospira sp., Borrelia sp., such as Borrelia burgdorferi), mollicutes (i.e. bacteria without cell wall, such as Mycoplasma sp.), acid-resistant bacteria (for example, Mycobacterium sp., such as Mycobacterium tuberculosum, Nocardia sp.). “Microbacterial organisms” also encompass fungi (such as yeasts and molds, for example, Candida sp., Aspergillus sp., Coccidioides sp., Cryptococcus sp., Histoplasma sp., Pneumocystis sp. Or Trichophyton sp.), Protozoa (for example, Plasmodium sp., Entamoeba sp., Giardia sp., Toxoplasma sp., Cryptosporidium sp., Trichomonas sp., Leishmania sp., Trypanosoma sp.) and archaea. Further examples of microbial organisms causing infectious disease that can be treated with the instant methods include, but are not limited to, Staphylococcus aureus (including methicillin-resistant S. aureus (MRSA)), Enterococcus sp. (including vancomycin-resistant enterococci (VRE), the nosocomial pathogen Enterococcus faecalis), food pathogens such as Bacillus subtilis, B. cereus, Listeria monocytogenes, Salmonella sp., and Legionella pneumophilia.

“Viral organism” or “virus”, which are used as equivalents herein, are small infectious agents that can replicate only inside the living cells of organisms. They include dsDNA viruses (e.g. Adenoviruses, Herpesviruses, Poxviruses), ssDNA viruses (e.g. Parvoviruses), dsRNA viruses (e.g. Reoviruses), (+) ssRNA viruses (e.g. Picornaviruses, Togaviruses, Coronaviruses), (−) ssRNA viruses (e.g. Orthomyxoviruses, Rhabdoviruses), ssRNA-RT (reverse transcribing) viruses, i.e. viruses with (+) sense RNA with DNA intermediate in life-cycle (e.g. Retroviruses), and dsDNA-RT viruses (e.g. Hepadnaviruses). Examples of viruses that can also infect human subjects include, but are not limited to, an adenovirus, an astrovirus, a hepadnavirus (e.g. hepatitis B virus), a herpesvirus (e.g. herpes simplex virus type I, the herpes simplex virus type 2, a Human cytomegalovirus, an Epstein-Barr virus, a varicella zoster virus, a roseolovirus), a papovavirus (e.g. the virus of human papilloma and a human polyoma virus), a poxvirus (e.g. a variola virus, a vaccinia virus, a smallpox virus), an arenavirus, a buniavirus, a calcivirus, a coronavirus (e.g. SARS coronavirus, MERS coronavirus, SARS-CoV-2 coronavirus (etiologic agent of COVID-19)), a filovirus (e.g. Ebola virus, Marburg virus), a flavivirus (e.g. yellow fever virus, a western Nile virus, a dengue fever virus, a hepatitis C virus, a tick-borne encephalitis virus, a Japanese encephalitis virus, an encephalitis virus), an orthomyxovirus (e.g. type A influenza virus, type B influenza virus and type C influenza virus), a paramyxovirus (e.g. a parainfluenza virus, a rubulavirus (mumps), a morbilivirus (measles), a pneumovirus, such as a human respiratory syncytial virus), a picornavirus (e.g. a poliovirus, a rhinovirus, a coxackie A virus, a coxackie B virus, a hepatitis A virus, an ecovirus and an enterovirus), a reovirus, a retrovirus (e.g. a lentivirus, such as a human immunodeficiency virus and a human T lymphotrophic virus (HTLV)), a rhabdovirus (e.g. rabies virus) or a togavirus (e.g. rubella virus). According to particular embodiments, the infectious disease to be treated is not HIV. According to alternative embodiments, the infectious disease to be treated is not a disease caused by a retrovirus. According to alternative embodiments, the infectious disease to be treated is not a viral disease.

This is equivalent as saying that methods of treating infectious disease are provided, comprising administering to a subject in need thereof a suitable dose of engineered cells as described herein (i.e. engineered cells comprising an exogenous nucleic acid molecule encoding two or more multiplexed RNA interference molecules, and optionally comprising a further nucleic acid molecule encoding a protein of interest), thereby improving at least one symptom. Particularly envisaged microbial or viral infectious diseases are those caused by the pathogens listed above.

These cells that are provided for use as a medicament can be provided for use in allogeneic therapies. I.e., they are provided for use in treatments where allogeneic ACT is considered a therapeutic option (wherein cells from another subject are provided to a subject in need thereof). According to specific embodiments, in allogeneic therapies, at least one of the RNA interference molecules will be directed against the TCR (most particularly, against a subunit of the TCR complex). According to alternative embodiments, these cells are provided for use in autologous therapies, particularly autologies ACT therapies (i.e., with cells obtained from the patient).

It is to be understood that although particular embodiments, specific configurations as well as materials and/or molecules, have been discussed herein for cells and methods according to present invention, various changes or modifications in form and detail may be made without departing from the scope and spirit of this invention. Importantly, the variations of the vectors as discussed in the different vector embodiments also apply to the engineered cells (as the vectors are suitable for expression in such cells), and vice versa: the various embodiments of the cells typically are linked to the vectors encoded in the cells. The following examples are provided to better illustrate particular embodiments, and they should not be considered limiting the application. The application is limited only by the claims.

### EXAMPLES

### Example 1. Optimisation of Multiplexing

Efficient processing of the miRNA from the transcribed RNA, by the DROSHA complex, is pivotal for efficient target knockdown. Our previous data showed that miRNA based shRNAs could efficiently be co-expressed with a CAR-encoding vector and processed by the miRNA machinery from the vector. It would further be desirable to generate a CAR expression vector, capable of co-expressing multiple miRNA based shRNAs (e.g. 2, 4, 6, 8 . . . ) from the same vector (FIG. 2). However, previous studies showed that co-expression of multiple miRNA-based shRNAs leads to loss of shRNA activity. Thus, for knocking down multiple targets from a single expression vector, efficient miRNA processing is important.

It was hypothesized that, to achieve optimal multiplexing and avoid recombination, it might be best to start from naturally occurring miRNA clusters, rather than multiplying a single miRNA scaffold. Naturally occurring miRNA clusters differ significantly in size and number of scaffolds present. As the goal is to use the multiplexed miRNA scaffolds for cloning vectors, we looked to identify clusters with a promising ratio of size over number of scaffolds. 13 of the identified clusters are listed in Table 1.

TABLE 1
Identification of micro-RNA clusters with indication of name, chromosomal location, size, location
within coding or non-coding sequences, and strand orientation. N indicates the number of microRNA
scaffolds present in the cluster; size/N is a division of those two columns and gives an indication
of the average size of a miRNA scaffold with interspersed sequences (linkers + other) in
said cluster. Clusters cl7.1, cl13.2, cl19.1, and clX.5: high expression in T cells.

| | Size | | | | | | |
|---|---|---|---|---|---|---|---|
| CLUSTERS | (bp) | Genomic position | N | miRNAs | Location | Strand | Size/N |
| cl1.4 | 280 | chr1: | 2 | miR-181b-1, miR-213 | Intergenic | + | 140 |
| | | 197094625-197094905 | | | | | |
| cl3.2 | 237 | chr3: | 2 | miR-15b, miR-16-2 | NFYC | + | 118.5 |
| | | 161605070-161605307 | | | | | |
| cl7.1 | 514 | chr7: | 3 | miR-25, miR-93, | DNM3(−) | − | 171 |
| | | 99529119-99529633 | | miR-106b | | | |
| cl11.1 | 302 | chr11: | 2 | miR-192, miR-194-2 | Predicted | − | 151 |
| | | 64415185-64415487 | | | gene | | |
| cl13.1 | 228 | chr13: | 2 | miR-16-1, miR-15a | mRNA | − | 114 |
| | | 49521110-49521338 | | | | | |
| cl13.2 | 786 | chr13: | 6 | miR-17, miR-92-1 | IARS2(−) | − | 131 |
| | | 90800860-90801646 | | | | | |
| cl17.3 | 249 | chr17: | 2 | miR-451, miR-144 | EST | − | 124.5 |
| | | 24212513-24212762 | | | | | |
| cl19.1 | 372 | chr19: | 3 | miR-24-2, miR-27a, | DALRD3 | − | 124 |
| | | 13808101-13808473 | | miR-23a | | | |
| cl19.2 | 312 | chr19: | 2 | miR-181c, miR-181d | SMC4L1 | + | 156 |
| | | 13846513-13846825 | | | | | |
| cl19.3 | 727 | chr19: | 3 | miR-99b, let-7e, | LARP7(−) | − | 242 |
| | | 56887677-56888404 | | miR-125a | | | |
| cl19.4 | 95751 | chr19: | 43 | miR-512-1, miR-519a-2 | EST/mRNA | + | 2226 |
| | | 58861745-58957496 | | | | | |
| clX.4 | 86225 | chrX: | 2 | miR-384, miR-325 | MCM7 | − | 43112 |
| | | 76056092-76142317 | | | | | |
| clX.5 | 900 | chrX: | 6 | miR-363, miR-106a | Intergenic | − | 150 |
| | | 133131074-133131974 | | | | | |

Two of those clusters (cl19.4 and clX.4, Table 1) are included for illustrative purposes, to show how divergent the size can be. These clusters are over 85000 bp and could immediately be excluded as they were too large for cloning. The most promising clusters were selected based on size and the number of miRNAs present in the clusters (the N in Table 1). Rather than total size alone, we evaluated the size divided by the number of miRNA scaffolds, to get an idea of the average miRNA scaffold+linker sequences. As a first cut-off, clusters with size/N lower than 250 were selected. As this yielded sufficient clusters and the goal was to express the vectors in engineered immune cells, it was decided to focus on clusters that are highly expressed in immune cells such as T cells. This led to a prioritization of 4 clusters (cl7.1, cl13.2, cl19.1, and clX.5, Table 1), all highly expressed in immune cells, with a total size less than 1000 bp. Furthermore, they all contained at least 3 miRNA scaffolds (clusters of N being at least three are promising to allow multiplexing of more than two miRNAs), and had an average size per scaffold of less than 200 bp, making them highly suitable for cloning (see Table 1): the miR17-92 cluster, the miR106a-363 cluster, the miR106b-25 cluster (three paralogous microRNA clusters) and the miR23a˜27a˜24-2 cluster.

1.1 Selection of a Suitable miRNA Cluster for Multiplexing

To evaluate whether the four miRNA clusters would be suitable for multiplexed expression of shRNAs, it was decided to transduce primary T cells from a healthy donor with retroviral vectors encoding a second generation CD19-directed CAR, a truncated CD34 selection marker along with different shRNAs introduced in the selected clusters. To allow comparison of a same number of shRNAs, and the effect of truncation of a cluster, fragments of the miR17-92 cluster and the miR106a-363 cluster were also used. The fragments were 3 or 4 consecutive miRNA scaffolds of the cluster, to allow comparison with the three miRNA scaffolds present in the other two clusters. The schematic design of such vectors is shown in FIG. 2.

Three identical shRNA target sequences were used for comparison, targeting CD247, B2M and CD52. When 4 miRNA scaffolds were used, TRAC was additionally targeted. For 6 miRNA scaffolds, the three targets were targeted twice, but with different target sequences. As a control, a repeated synthetic shRNA scaffold was used, the miR196a2 scaffold, which was shown previously to be excellent for single shRNA knockdown, as well as suitable for multiplexed knockdown (WO2020/221939). This control was used with 3 and 4 shRNAs.

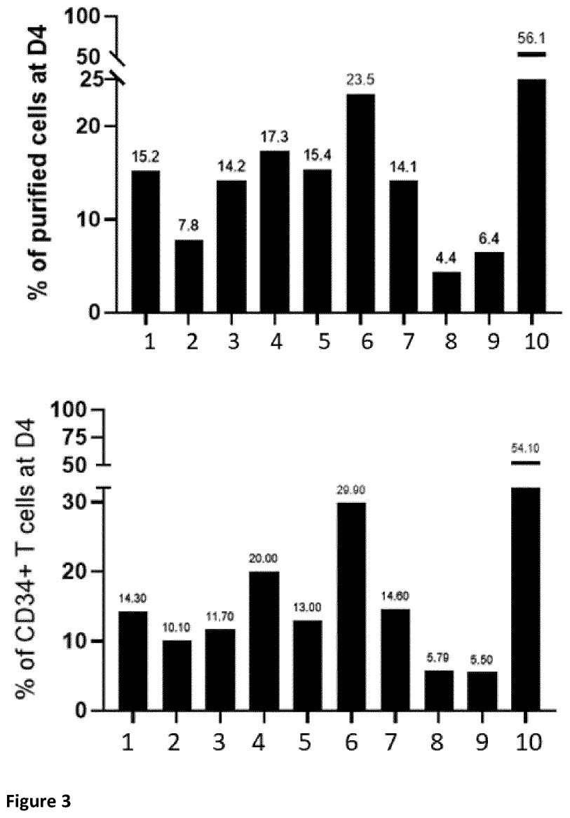

Despite the different size of the constructs, vector titres were only slightly affected by the amount of shRNAs present (data not shown). However, in every constellation, the use of different scaffolds from the natural miRNA clusters increases the transduction efficiency compared to repeated identical scaffolds (here the miR-196a2 scaffold), as shown in FIG. 3.

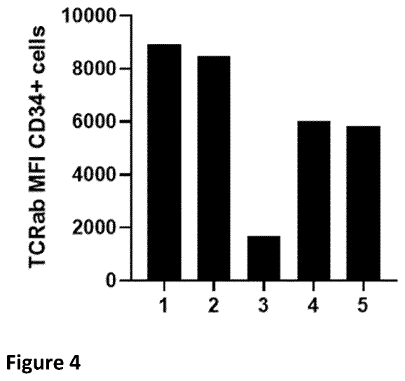

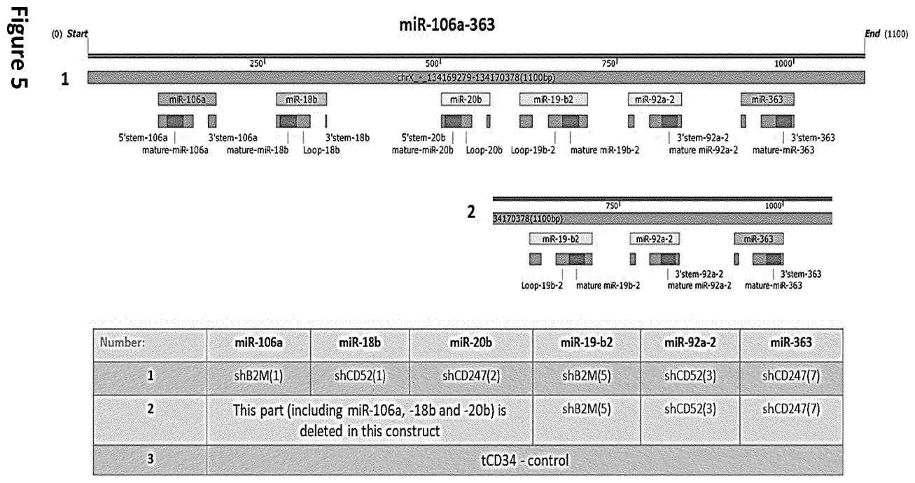

T cell fold increase from transduction to harvest did not differ significantly between the constructs (neither between the clustered scaffolds, nor between the clustered scaffolds and the repeated single scaffolds). However, the knockdown efficiency did differ between the constructs. Although all clusters achieved knockdown to some extent, there was a clear difference between the clustered scaffolds, with the scaffolds from the miR-106a-363 cluster achieving the best and most consistent knockdown and those of the miR23a˜27a˜24-2 cluster being least effective. In FIG. 4, an example is shown comparing TCR expression of a control without shRNA, or with shRNA in a miR23a˜27a˜24-2 clustered scaffold, or in a miR106a-363 clustered scaffold or a fragment thereof. The increased knockdown observed with the full scaffold can be explained by the fact that CD247 is targeted twice in this construct. As a result of these experiments, the scaffolds of the miR-106a-363 cluster were selected for further evaluation.

### Example 2. Multiplexing Using the Scaffolds of the miR-106a-363 Cluster

The feasibility of multiplexing up to six shRNAs was assessed in hard to transduce primary immune cells. To assess this, primary T cells were transduced with retroviral vectors encoding a second generation CD19 CAR containing either 3×shRNAs or 6×shRNAs targeting CD247, β2m and CD52 introduced in the miR-106a-363 cluster. The design of the vector is shown in FIG. 5.

Briefly, primary T cells from a healthy donor were transduced with retroviral vectors encoding a second generation CD19-directed CAR, a truncated CD34 selection marker along with 3 shRNAs targeting CD247, B2M and CD52, introduced in the last three miRs of the 106a-363miRNA cluster (miR-19b2, miR-92a2 and miR-363), or 6 shRNAs targeting the same three genes in the 6 miR scaffolds of the cluster (in this case the two shRNAs targeting CD247 were different). Concisely, shRNAs expressed as a 6-plex, 3-plex or no shRNA (tCD34) as control. Two days after transduction, cells were enriched using CD34-specific magnetic beads, and further amplified in IL-2 (100 IU/mL) for 6 days. mRNA expression of CD247, B2M and CD52 was assessed by qRT-PCR using cyclophilin as house-keeping gene.

Results are shown in FIG. 6. Multiplexed shRNAs yielded efficient RNA knock-down levels for all targeted genes. Incorporation of six multiplexed shRNAs (two shRNAs against each protein target) resulted in higher RNA knock-down levels compared to three multiplexed shRNAs (one shRNA against each protein target) (FIG. 6).

### Example 3. Optimisation of Individual Scaffolds of the miR-106a-363 Cluster

Although the initial data were already promising and showed multiplexing can be achieved when scaffolds from the miR-106a-363 cluster are used, further studies were done to see whether individual scaffolds could be modified to improve knockdown of the selected target. Since it stands to reason that the natural scaffold already was under evolutionary selection pressure to accommodate knockdown (meaning that the lower and upper stem regions were at least partly optimized by evolution), it was decided to first evaluate different target sequences to improve target downregulation, as these had not yet been optimized. In first instance, the same target proteins were selected.

As it had been described before that the processivity of each miRNA/shRNA may depend on and be influenced by that of others in the cluster (Bofill-De Ros and Gu, 2016), it was decided to test the scaffolds with different target sequences as part of the whole cluster, but with irrelevant sequences in the other scaffold sequences (to not influence target downregulation).

Results for downregulation of CD247 in the miR-20b scaffold are shown in FIG. 7. The initial scaffold sequence already resulted in about 50% downregulation. All other target sequences tested also resulted in successful knockdown of the target, but some achieved much more than 50% knockdown. In other words, by selecting the target sequence a maximally effective knockdown could be achieved, no further engineering of the miR-20b scaffold was necessary.

Similar results were obtained for the miR-106a scaffold sequence, using different sequences for the B2M target (data not shown). To rule out that the effect is linked to the specific target sequence-scaffold combination, the B2M target sequences were also tested in the miR-20b scaffold. Although there was some minor variation in terms of knockdown efficiency, the three target sequences achieving highest knockdown in the miR-106a scaffold also achieved highest knockdown when used in the miR-20b scaffold. This means that once an effective target sequence is identified, it can be used across scaffolds.

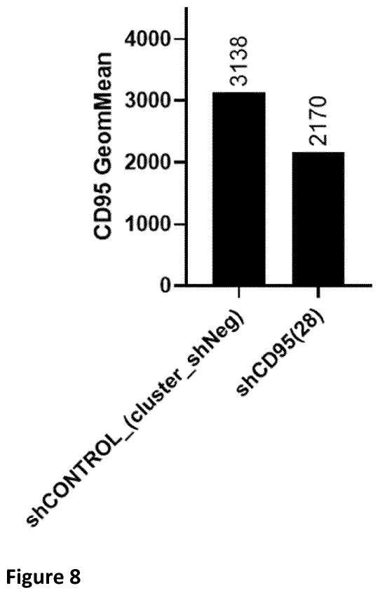

For the miR-18b scaffold, optimization of a shRNA against CD95 was undertaken. However, after testing 31 target sequences, the best knockdown achieved was about 30% (see FIG. 8). Although this knockdown is non-negligible, it is considerably less effective than the over 75% knockdown consistently obtained for other scaffolds. When comparing the miR-18b scaffold with that of miR-106a or miR-20b (FIG. 9), it is apparent that this scaffold contains more mismatches in the target sequence/upper stem region (three versus one), as well as a bulge near the end of the upper stem. As high knockdown was achieved with the other scaffold sequences, it was hypothesized that reducing the number of mismatches and/or removing the bulge could potentially improve the knockdown efficiency.

The 5 different constructs evaluated are shown in FIG. 10A, the results in FIG. 10B. Remarkably, deleting even a single mismatch or bulge drastically improves the knockdown efficiency. When only the single mismatch that occurs as well in the miR-106a or miR-20b scaffold is kept, the knockdown efficiency increases from about 30% to over 60% for the same target sequence. Thus, although the miR-18b scaffold sequence can be used as such, knockdown efficiency can be significantly increased by reducing the number of mismatches or the bulge. For further examples herein, the construct 28.5 is used, where the bulge at position 15-16 and the mismatches at position 20 and 25 have been removed, but the mismatch at position 29 is retained. Of note, the removal of the mismatches is by adapting the passenger sequence, as the target sequence needs to match.

### Example 4. Evaluation of Target Sequence Length

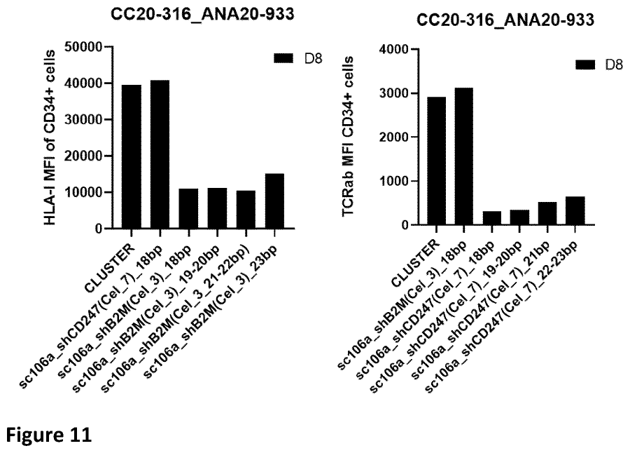

The natural target sequences found in the miR-106a-363 cluster are typically quite long (22-23 bp). To evaluate whether these could be shortened, different lengths of target sequence (one directed against CD247, one against B2M) were inserted in the scaffold and evaluated for knockdown efficiency. Shortening of the sequence was done by replacing nucleotides at the 3′ end of the target sequence with those found in the natural scaffold. Results for the miR-106a scaffold are shown in FIG. 11. It can be seen that shorter sequences, down to 18 bp, work as well as, and maybe even better than, the maximal length. Similar results were obtained for the miR-20b scaffold (not shown). For most experiments, it was decided to work with a target sequence of 20 bp (as indicated in FIG. 9).

### Example 5. Evaluation of Combination of Individual Scaffolds Outside the Cluster Context

It is generally accepted that in miRNA clusters, a lot of flanking sequence determinants as well as the presence of other clusters is believed to be important to achieve downregulation. However, earlier experiments by us had shown this is not always the case.

Indeed, in order to optimize activity of two co-expressed shRNAs, we earlier hypothesized that not only the size, but also the sequence of the linker between two miRNA-based shRNAs, as well as the miRNA scaffold would affect shRNA activity. In order to optimize the shRNA processing, we assessed the impact of different shRNA linkers on the knockdown of two target genes, CD247 (CD37) and CD52. Linkers from 0 to 92 bp were used, but apart from the construct lacking any spacer between the two hairpins, which showed a slightly lower knockdown activity for TCR (but not for CD52) compared to the other constructs, the linker did not appear to affect the knockdown efficacy. Importantly, even the construct without linker still worked very well in reducing expression for both shRNAs (data not shown). Although these experiments were done with a miR-196a2 scaffold, initial experiments indicated the linkers of the miR-106a-363 cluster could be significantly reduced as well.

To evaluate whether the processivity and activity of the individual scaffolds were influenced by the presence of others in the cluster, it was decided to test the scaffolds in different permutations. To this end, non-consecutive scaffolds were selected (to eliminate the effect of neighbouring scaffolds in the cluster): miR-106a and miR-20b. Further, duplexes and triplexes were created rather than using all six miRNA scaffolds in the cluster (contrary to Example 2). The miR-106a-miR-18b-miR-20b triplex was also created, corresponding to the first three scaffolds in the miR-106a˜363 cluster, to evaluate whether there was a cluster context effect. For duplexes, the genes targeted were B2M and CD247. For triplexes, CD95 was added.

In summary, the following constructs were made:

### Duplexes:

miR-106a (targeting B2M)-miR-20b (targeting CD247)
miR-20b (targeting CD247)-miR-106a (targeting B2M)
miR-20b (targeting B2M)-miR-20b (targeting CD247)
miR-106a (targeting B2M)-miR-106a (targeting CD247)

### Triplexes:

miR-20b (targeting B2M)-miR-20b (targeting CD95)-miR-20b (targeting CD247)
miR-106a (targeting B2M)-miR-106a (targeting CD95)-miR-106a (targeting CD247)
miR-106a (targeting B2M)-miR-18b (targeting CD95)-miR-20b (targeting CD247)

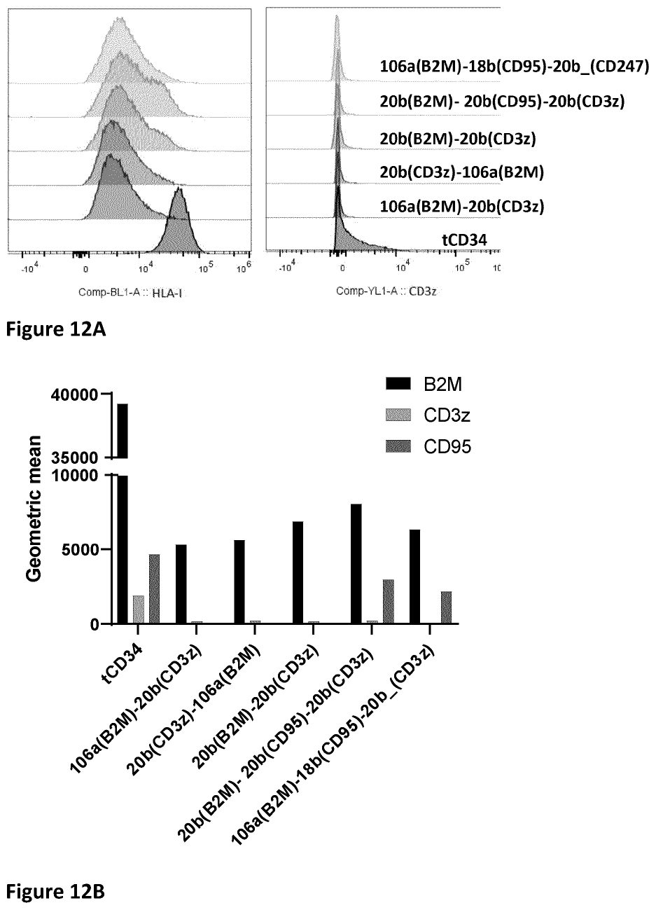

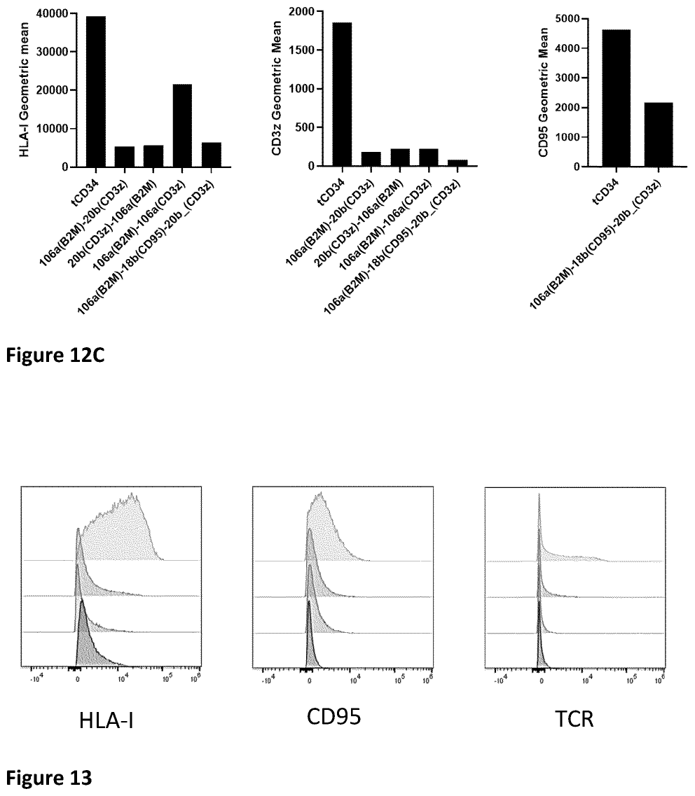

Results are shown in FIG. 12A-C. As shown in FIG. 12, all of the duplexes evaluated were very efficient in downregulating both CD247 and B2M. The CD247 knockdown in particular proved to be very efficient, leading to barely detectable levels of CD3Z. As B2M is far more abundant, the knockdown was not expected to be complete, but a reduction of over 80% in B2M levels was consistently achieved. Remarkably, the level of downregulation is identical regardless of the order of the scaffolds in the duplex.

When multiplexing identical shRNAs, it is well known that recombination presents an issue, resulting in much lower expression and ultimately lower knockdown levels. This was exactly the reason to evaluate a combination of different scaffolds. Nevertheless, two and three identical scaffolds were tested to see whether this was practically feasible. All of the duplexes with identical scaffolds, and the miR-20b triplex scaffold achieved levels of transduction comparable to duplexes or triplexes with different scaffolds, and all were above 15%. However, the miR-106a triplex scaffold yielded very low transduction levels (less than 2%) and was not further evaluated. Duplexes of miR-20b scaffolds achieved identical levels of knockdown for the targets as duplexes with non-identical scaffolds (FIG. 12A-B). Duplexes of the miR-106a scaffold achieved the same downregulation for CD3Z, but were slightly less effective in B2M knockdown, although levels were reduced by approximately 50%, indicating that these scaffolds can be duplicated and still achieve high knockdown (FIG. 12C). Remarkably, the miR-20b triplex scaffold achieved knockdown levels that are comparable to a triplex with three different scaffolds, although the use of three different scaffolds does yield slightly better knockdown for each target gene, indicating there is some loss of efficacy (FIG. 12A-B). The triplex scaffold with three different miRNA scaffolds achieves identical downregulation of the targets as the duplexes. Additionally, CD95 is downregulated over 50% (FIG. 12B-C), which is in line with the results of this target sequence when used in the cluster setting (FIG. 10B).

These experiments show that the scaffolds can very well be used independently, outside the context of the cluster. The order of the scaffolds does not seem to be important to achieve the desired knockdown, and not all scaffolds of the cluster need to be present to achieve knockdown. Indeed, a single scaffold is sufficient, and it can be duplicated without loss of activity. Although it was shown that the miR-20b can be used as a triplex, this seems to be slightly less efficient than using different scaffolds. Still, considering there are six different scaffold sequences in the miR-106a-363 cluster and these can be duplicated without loss of effect, multiplexed downregulation of up to 12 targets is in principle feasible.

### Example 6. Optimisation of Individual Scaffolds by Making Chimeric Scaffolds

As alternative of removing mismatches or bulges in individual scaffolds (see Example 3), it was reasoned that the knockdown efficiency of individual scaffolds with mismatches and/or bulges (such as miR-18b) could also be improved not just by mutating the scaffold by removing mismatches, but also by substituting the upper stem/loop region with that of a scaffold that has high knockdown efficiency. Indeed, when the upper stem/loop region of either the miR-106a scaffold or the miR-20b scaffold was used to replace the upper stem/loop region of the miR-18b scaffold, the knockdown efficiency of the selected target was significantly increased (data not shown). As there are several conserved scaffolds within the three miR-17 paralog clusters, it was decided to evaluate this for other scaffold sequences as well. Results showed that, indeed, scaffolds with few mismatches and bulges that resemble the miR-106a, mir-20b or miR-17 scaffold could indeed be used. I.e., the upper stem/loop region of a scaffold from the miR-17 family (a miR-17 scaffold, a miR-20a scaffold, a miR-106a scaffold, a miR-20b scaffold, a miR-106b scaffold, and a miR-93 scaffold) could be fused to a lower stem of a scaffold in either the miR106a-363, miR17-92, miR106b-25 cluster to maintain or increase knockdown efficiency. By way of example, FIG. 13 shows the effect of changes in the scaffolds on knockdown efficacy. To this end, 4 different constructs were tested: a negative control containing only a CAR T without shRNA, and 3 CAR T cells with shRNA: one duplex with miR-106a scaffold (targeting B2M) and miR-20b scaffold (targeting CD247), a triplex with miR-106a scaffold (targeting B2M)-miR-18b scaffold (targeting CD95)-miR-20b scaffold (targeting CD247), wherein the loop region of miR-18 has been altered (see Example 3), and a triplex with miR-106a scaffold (targeting B2M)-miR-18b scaffold (targeting CD95)-miR-20b scaffold (targeting CD247), wherein the upper stem/loop region of scaffold miR-18b was replaced with the upper stem/loop region of the miR-17 scaffold. As seen in FIG. 13, knockdown of HLA-I and TCR is achieved in all three constructs, and knockdown of CD95 is achieved in the triplex constructs. However, the chimeric scaffold construct achieved a higher degree of knockdown for CD95. At the same time, it turns out that the change in the scaffold targeting CD95 also induce changes in the expression of HLA-I (FIG. 13, left panel) while both scaffold and target sequence remain unaltered, thus showcasing changes in the microprocessing of the cluster.

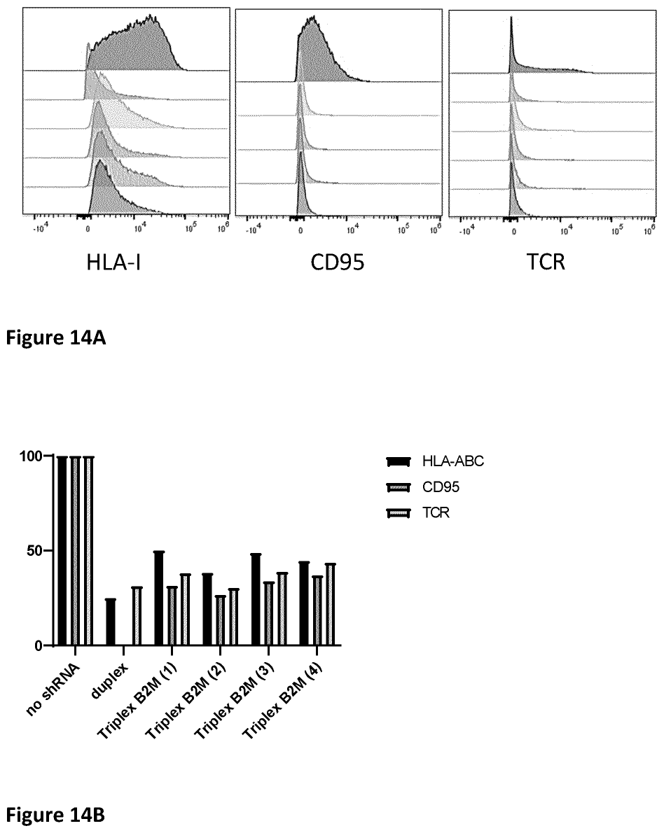

To optimize this further, it was decided to keep the microprocessing constant by retaining the scaffold sequences of the latter construct (i.e., triplex with miR-106a scaffold (targeting B2M)-miR-18b scaffold (targeting CD95)-miR-20b scaffold (targeting CD247), wherein the upper stem/loop region of scaffold miR-18b was replaced with the upper stem/loop region of the miR-17 scaffold). To ensure that the microprocessing did not depend on the target sequence, different target sequences against B2M were assessed. Results are shown in FIG. 14. Four different B2M target sequences in a triplex scaffold were tested against the duplex with miR-106a scaffold (targeting B2M) and miR-20b scaffold (targeting CD247) and a negative control containing CAR T cells without shRNA. All triplex constructs achieved good knockdown of all three targets (FIG. 14A, B). While the HLA-I expression shows some variability due to the different target sequences, the knockdown of the other two targets remained constant. All four sequences tested achieved at least 50% knockdown, while retaining the TCR knockdown and additionally achieving high knockdown of CD95.

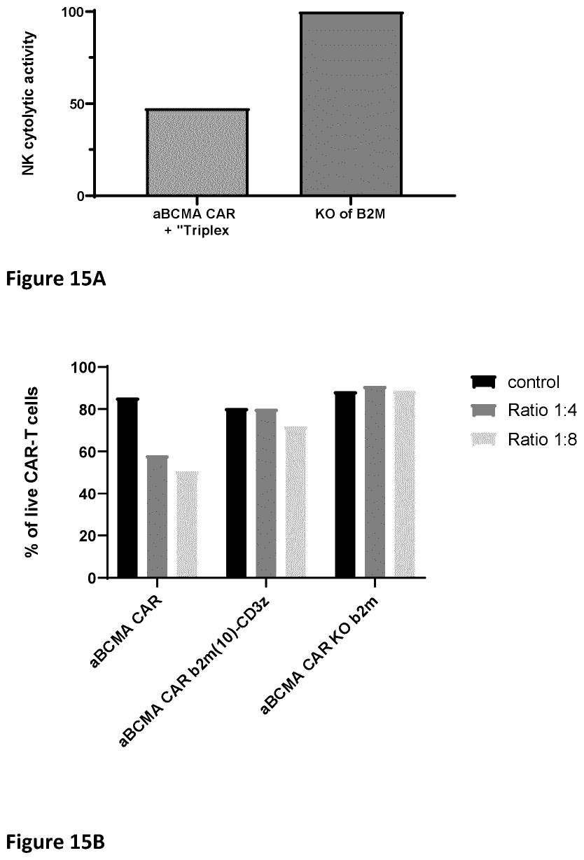

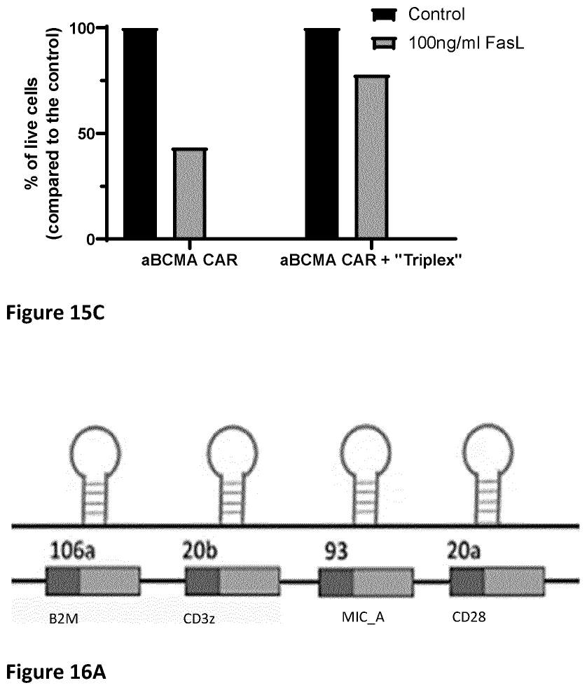

To check whether the knockdown of the sequence also yielded functional results, different functional assays were done with the BCMA CAR T cells additionally transduced with the triplex scaffold. Results are shown in FIG. 15. By targeting B2M in adoptive T cells, HLA-I should be downregulated, thereby inhibiting T cell allorecognition and preventing host versus graft response. However, if HLA is completely depleted, cells become prey to NK mediated cell killing, which is why gene edited cell therapies typically require co-expression of HLA-E. We postulated that the knockdown of B2M would prove beneficial in this regard, as HLA-I is downregulated, but not to the extent that cells are completely eliminated by NK-mediated cytolysis. As shown in FIG. 15A, when cells were coincubated with NK cells, the downregulation of B2M achieved in the triplex shRNA scaffold protects BCMA CAR T cells against NK mediated killing. As comparison, a BCMA CAR-T with B2M knockout with Crispr/Cas9 was used and was lysed completely by the allogeneic NK cells. Importantly, as shown in FIG. 15B, shRNA against B2M inhibits T cell allorecognition similar to B2M knockout, with 80% of the cells remaining alive compared to 50% in the CAR without shRNA. Finally, to test the effect of the shRNA against CD95 (the Fas receptor), cells were incubated with 100 ng/ml Fas ligand. As seen in FIG. 15C, shRNA against CD95 (in the triplex scaffold) protects against FasL mediated apoptosis.

These results show that shRNA can successfully be multiplexed when a chimeric scaffold is included in the cluster.

### Example 7. Optimisation of Clusters by Making Chimeric Clusters

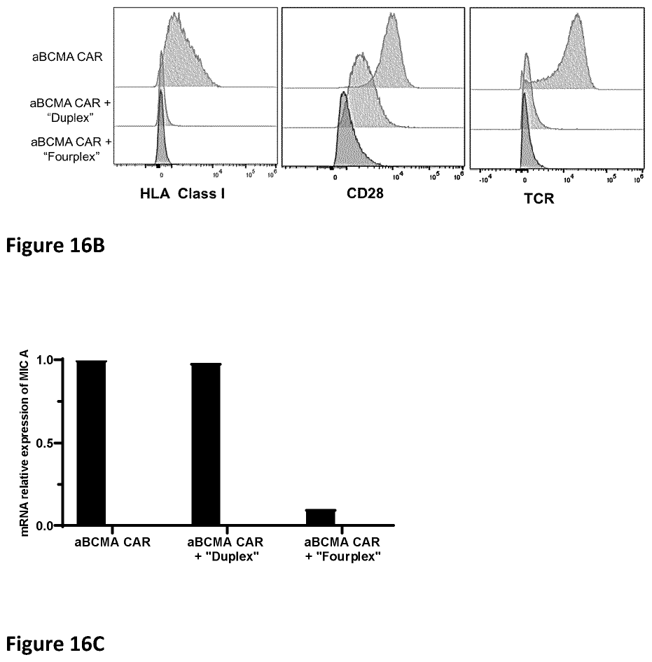

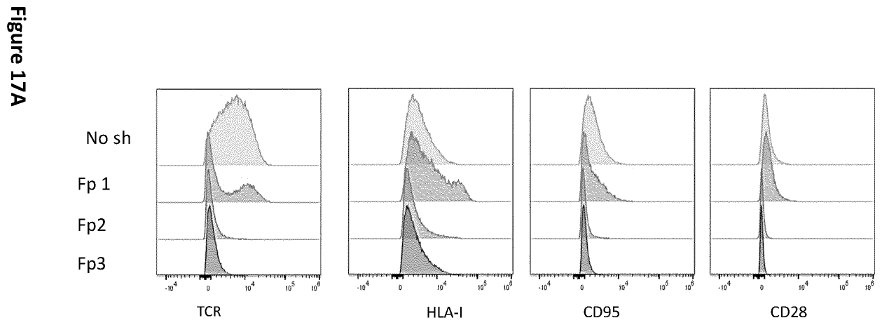

Since the upper stem and loop regions of miR-17 family scaffolds proved beneficial in optimizing knockdown efficiency of the miR-106a-363 cluster, it was evaluated whether we could create a multiplex cluster by using only scaffolds of the miR-17 family. To this end, a fourplex shRNA cluster was designed containing 4 shRNA scaffolds derived from the three different miR17-92 paralogue clusters: miR-106a and miR-20b from the miR-106a-363 cluster; miR-93 from the miR-106b-25 cluster and miR-20a from the miR-17-92 cluster. Target genes were B2M in miR-106a, CD3 zeta in miR-20b (as described before), MICA (a NKG2D ligand) in miR-93 and CD28 in miR-20a. A schematic representation of the construct is shown in FIG. 16A. We used wild type/naturally flanking sequences as linkers (cf Example 5), no additional restriction sites were inserted between the scaffolds to minimize the risk of altered microprocessing. Knockdown was compared to a negative control without shRNA and the duplex of the same miR-106a scaffold (targeting B2M) and miR-20b scaffold (targeting CD247). As shown in FIG. 16B, left and right panels, knockdown of TCR and HLA class I is similar in the duplex and fourplex constructs. In addition, the fourplex succeeds in knocking down expression of CD28 (FIG. 16B, middle panel), and knocking down expression of MICA (FIG. 16C). Thus, while the context (and thus the processing) of the scaffolds has changed compared to the wildtype clusters, it is clear that miR-17 family scaffolds can be combined to achieve multiplex knockdown. Moreover, this is without encountering recombination, as demonstrated by the high levels of knockdown achieved.

To further corroborate this, it was decided to add a further scaffold. A fiveplex scaffold was created by adding a miR-17 scaffold to the fourplex scaffold shown in FIG. 16A. The target sequence of this scaffold was CD95.

This fiveplex scaffold was compared to two other fiveplex scaffolds: one in which the target sequences were kept identical, but two (MICA and CD28) were exchanged from scaffold to assess positional effects, and one in which a different B2M and CD3 zeta sequence were used that were optimized for a different and unrelated scaffold. As microprocessing for unrelated scaffolds can be different, we wanted to check whether using unoptimized sequences is a valid strategy or not.
Fiveplex 1: miR-106a (targeting B2M with unoptimized sequence)-miR-20b (targeting CD247 with unoptimized sequence)-miR-93b (targeting CD28)-miR-20a (targeting MICA)-miR-17 (targeting CD95)
Fiveplex 2: miR-106a (targeting B2M)-miR-20b (targeting CD247)-miR-93b (targeting CD28)-miR-20a (targeting MICA)-miR-17 (targeting CD95)
Fiveplex 3: miR-106a (targeting B2M)-miR-20b (targeting CD247)-miR-93b (targeting MICA)-miR-20a (targeting CD28)-miR-17 (targeting CD95)

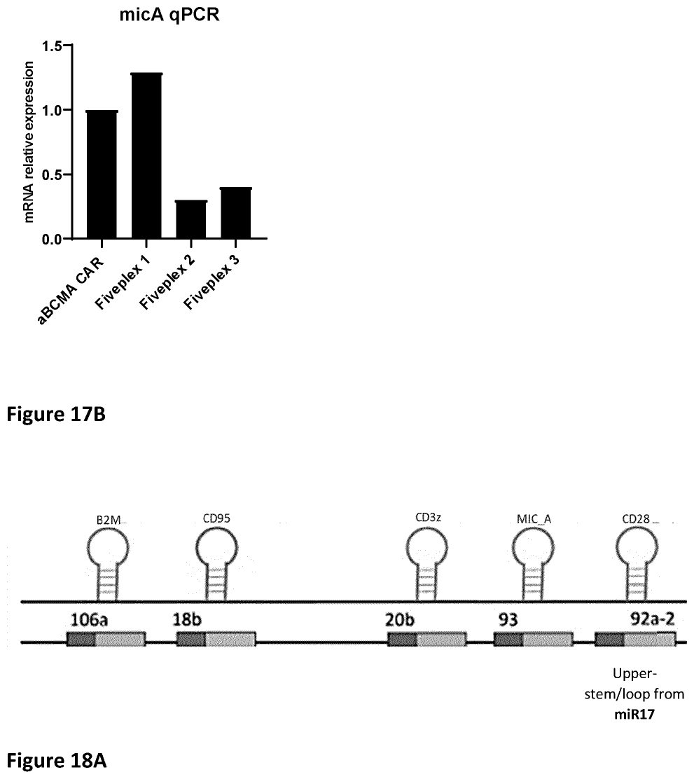

Results are shown in FIG. 17. Fiveplex 2 and 3 achieve knockdown of all 5 target genes (FIGS. 17A and B), which indicates that target sequences can be switched within related scaffolds without affecting the knockdown or the microprocessing of the cluster. However, this appears only true for target sequences optimized for related scaffolds. The knockdown of TCR is less efficient in fiveplex 1, and knockdown of HLA-I is almost non-existent. Furthermore, also knockdown of CD95 and CD28 appears less efficient (FIG. 17A), and knockdown of MICA is unsuccessful (FIG. 17B), even though the scaffolds and sequences used for these four targets were identical to those in fiveplexes 2 and 3. This points again to changes in microprocessing that affect knockdown of multiple sequences (see also FIG. 13). Thus, a sequence that works in a scaffold from the miR-17 family cluster can be used in different scaffolds from the miR-17 family, even in a multiplex setting. However, sequences that can be used in other scaffolds cannot automatically be used in a miR-17 family cluster.

### Example 8. Optimisation of Clusters by Combining Chimeric Clusters with Chimeric Scaffolds

As shown in Examples 6 and 7, optimal multiplexing results are achieved when suboptimal scaffolds are modified by making them chimeric with a miR-17 family upper stem/loop region (Example 6) or by combining several miR-17 family scaffolds from different miR-17-92 paralog clusters (Example 7). Next, it was assessed whether these two strategies could be combined to achieve even higher multiplexing.

First, a fiveplex chimeric construct was designed that looked as follows:

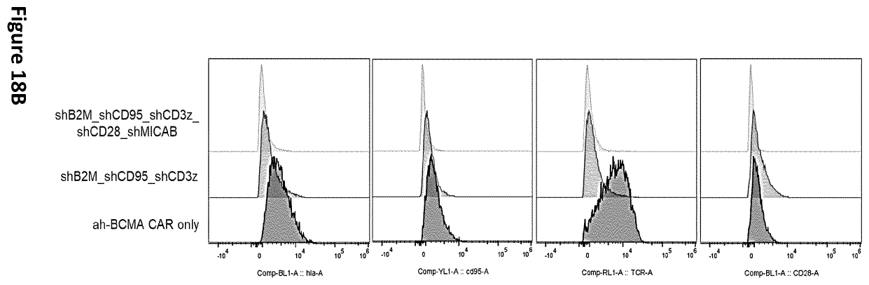

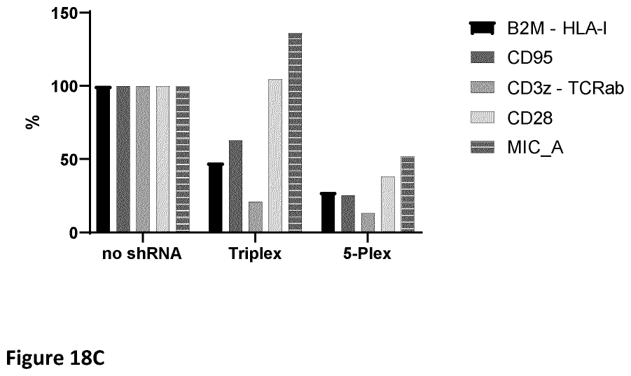

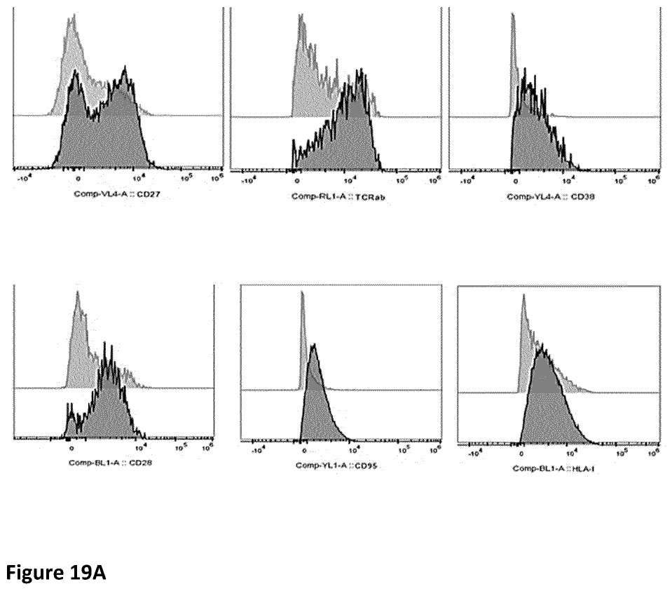

miR-106a (targeting B2M)-optimized miR-18b (targeting CD95)-miR-20b (targeting CD247)-miR-93b (targeting MICA)-chimeric miR-92a2 with an upper stem/loop region from miR-17 (targeting CD28). See Scheme in FIG. 18A.

In essence, this is the triplex construct described in Example 6 fused to the miR-93b scaffold (as done in Example 7) with an additional chimeric scaffold with a lower stem from miR-92a2 (a scaffold from the miR-106a-363 cluster) and an upper stem and loop from miR-17.

When this fiveplex construct was compared to the triplex construct (only consisting of miR-106a (targeting B2M)-optimized miR-18b (targeting CD95)-miR-20b (targeting CD247)), it can be seen that the fiveplex is at least as efficient as the triplex in achieving knockdown of the target genes. The upper and middle histograms in FIG. 18B show clear reduction compared to the lower control histograms. FIG. 18C shows the same results as FIG. 18B, but as relative MFI. The triplex successfully knocks down three target genes, the fiveplex manages to downregulate 5 genes, all to a similar extent.

Finally, a sixplex cluster was designed, by adding a further chimeric scaffold, this time with the miR-363 lower stem and the miR-20a upper stem and loop.

The design was as follows:

miR-106a (targeting CD38)-optimized miR-18b (targeting CD95)-miR-20b (targeting CD247)-miR-93b (targeting B2M)-chimeric miR-92a2 with an upper stem/loop region from miR-17 (targeting CD28)-chimeric miR-363 with an upper stem/loop region from miR-20a (targeting CD27).

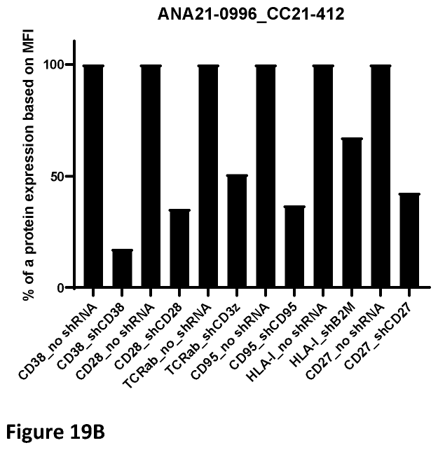

As can be seen, the B2M targeting sequence was tested in a different, non-adjacent scaffold. As shown in FIG. 19A, all six genes were knocked down compared to a control without shRNA. FIG. 19B shows the same results as FIG. 19A, but as relative MFI. All targets achieved over 50% knockdown, with the exception of B2M, likely due to the fact of the abundance of that target.

We consistently showed multiplexed knockdown when using miRNA scaffolds from a miR-17 family cluster, a knockdown which can be further improved by making chimeric scaffolds using upper stem/loop regions from a miR-17 family scaffold; and/or by making chimeric clusters and using miR-17 family scaffolds from different paralog clusters.

It will be recognised that the invention is not limited to the specific details described herein which are given by way of example only, and various modifications and alterations are possible within the scope of the invention.

### REFERENCES

Bofill-De Ros X, Gu S. Guidelines for the optimal design of miRNA-based shRNAs. Methods. 2016 Jul. 1; 103:157-66.
Chumakov S P, Kravchenko J E, Prassolov V S, Frolova E I, Chumakov P M. Efficient downregulation of multiple mRNA targets with a single shRNA-expressing lentiviral vector. Plasmid. 2010 May; 63 (3): 143-9.
Fowler D K, Williams C, Gerritsen A T, Washbourne P. Improved knockdown from artificial microRNAs in an enhanced miR-155 backbone: a designer's guide to potent multi-target RNAi. Nucleic Acids Res. 2016 Mar. 18; 44 (5): e48.
Giering J C, Grimm D, Storm T A, Kay M A. Expression of shRNA from a tissue-specific pol II promoter is an effective and safe RNAi therapeutic. Mol Ther. 2008 September; 16 (9): 1630-6.
Grimm D, Streetz K L, Jopling C L, Storm T A, Pandey K, Davis C R, Marion P, Salazar F, Kay M A. Fatality in mice due to oversaturation of cellular microRNA/short hairpin RNA pathways. Nature. 2006 May 25; 441 (7092): 537-41.
Jiang Z, Han Y, Cao X. Induced pluripotent stem cell (iPSCs) and their application in immunotherapy. Cell Mol Immunol. 2014 January; 11 (1): 17-24.
Lebbink R J, Lowe M, Chan T, Khine H, Wang X, McManus M T. Polymerase II promoter strength determines efficacy of microRNA adapted shRNAs. PLOS One. 2011; 6 (10): e26213.
Moore C B, Guthrie E H, Huang M T, Taxman D J. Short hairpin RNA (shRNA): design, delivery, and assessment of gene knockdown. Methods Mol Biol. 2010; 629:141-58.
Taxman D J, Livingstone L R, Zhang J, Conti B J, Iocca H A, Williams K L, Lich J D, Ting J P, Reed W. Criteria for effective design, construction, and gene knockdown by shRNA vectors. BMC Biotechnol. 2006 Jan. 24; 6:7.
Themeli M, Rivière I, Sadelain M. New cell sources for T cell engineering and adoptive immunotherapy. Cell Stem Cell. 2015 Apr. 2; 16 (4): 357-66.

1. A nucleic acid molecule comprising at least one RNA interference molecule with an engineered scaffold, wherein the engineered scaffold comprises a lower stem region and an upper stem/loop region, and wherein the lower stem region of the scaffold is that of a miR scaffold from the miR-17 family cluster, and wherein at least part of the upper stem/loop region of the scaffold has been engineered to differ from a wild-type sequence.

2. The nucleic acid molecule according to claim 1, wherein the lower stem region of the engineered scaffold is selected from a miR-17 scaffold, a miR-18a scaffold, a miR-19a scaffold, a miR-20a scaffold, a miR-19b-1 scaffold, a miR-92-1 scaffold, a miR-106a scaffold, a miR-18b scaffold, a miR-20b scaffold, a miR-19b-2 scaffold, a miR-92-2 scaffold, a miR-363 scaffold, a miR-106b scaffold, a miR-25 scaffold, and a miR-93 scaffold.

3. The nucleic acid molecule according to claim 1, wherein the engineered scaffold is a chimeric scaffold and wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold.

4. The nucleic acid molecule according to claim 1, wherein the at least one RNA interference molecule are at least two multiplexed RNA interference molecules.

5. A nucleic acid molecule comprising at least two RNA interference molecules with different scaffolds, wherein the at least two different scaffolds have a lower stem region of a miR scaffold from the miR-17 family cluster; and wherein
at least one RNA interference molecule has a chimeric scaffold
wherein at least part of the upper stem/loop region is not from the same miR scaffold as the lower stem region, and
wherein the at least part of the upper stem/loop region is selected from a miR-17 scaffold, a miR-20a scaffold, a miR-106a scaffold, a miR-20b scaffold, a miR-106b scaffold, and a miR-93 scaffold; and/or wherein
the scaffolds from the at least two RNA interference molecules are a miR-17 family scaffold from different miR-17 family clusters.

6. A vector suitable for expression in engineered immune cells comprising a nucleic acid molecule according to claim 1.

7. An engineered cell comprising:
a first exogenous nucleic acid molecule encoding a protein of interest, and
a second nucleic acid molecule comprising at least one RNA interference molecule with an engineered scaffold, wherein the lower stem region of the scaffold is that of a miR scaffold from the miR-17 family cluster, and wherein at least part of the upper stem/loop region of the scaffold has been engineered to differ from the wild-type sequence.

8. An engineered cell comprising:
a first exogenous nucleic acid molecule encoding a protein of interest, and
a second nucleic acid molecule comprising at least two RNA interference molecules with different scaffolds, wherein the at least two different scaffolds have a lower stem region of a miR scaffold from the miR-17 family cluster; and wherein
at least one RNA interference molecule has a chimeric scaffold wherein at least part of the upper stem/loop region is not from the same miR scaffold as the lower stem region, and wherein the at least part of the upper stem/loop region is selected from a miR-17 scaffold, a miR-20a scaffold, a miR-106a scaffold, a miR-20b scaffold, a miR-106b scaffold, and a miR-93 scaffold; and/or wherein
the scaffolds from the at least two RNA interference molecules are a miR-17 family scaffold from different miR-17 family clusters.

9. The engineered cell of claim 7, which is an engineered immune cell.

10. The engineered cell of claim 9, wherein the engineered immune cell is selected from a T cell, a NK cell, a NKT cell, a macrophage, a stem cell, a progenitor cell, and an iPSC cell.

11. The engineered cell of claim 7, wherein the protein of interest is a receptor, particularly a chimeric antigen receptor or a TCR.

12. The engineered cell of claim 7, wherein the at least one RNA interference molecule is at least two multiplexed RNA interference molecules under control of one promoter.

13. The engineered cell of claim 12, wherein the at least two multiplexed RNA interference molecules are at least three multiplexed RNA interference molecules.

14.-15. (canceled)

16. The engineered cell of claim 8, which is an engineered immune cell.

17. The engineered cell of claim 16, wherein the engineered immune cell is selected from a T cell, a NK cell, a NKT cell, a macrophage, a stem cell, a progenitor cell, and an iPSC cell.

18. The engineered cell of claim 8, wherein the protein of interest is a receptor, particularly a chimeric antigen receptor or a TCR.

19. The engineered cell of claim 8, wherein the at least one RNA interference molecule is at least two multiplexed RNA interference molecules under control of one promoter.

20. The engineered cell of claim 19, wherein the at least two multiplexed RNA interference molecules are at least three multiplexed RNA interference molecules.

21. A method of treating cancer, comprising administering to a subject in need thereof a suitable dose of cells according to claim 7, thereby improving at least one symptom.

22. A method of treating cancer, comprising administering to a subject in need thereof a suitable dose of cells according to claim 8, thereby improving at least one symptom.
